SEQUENCE LISTING (7 sequences)

SEQ ID NO: 1 - DNA, 21 bp, Artificial Sequence
  Synthetic primer
agaaagcgatggctaactatg

SEQ ID NO: 2 - DNA, 16 bp, Artificial Sequence
  Synthetic primer
gcgtggactaccaggg

SEQ ID NO: 3 - DNA, 21 bp, Artificial Sequence
  Synthetic primer
aataagccccggctaactatg

SEQ ID NO: 4 - DNA, 28 bp, Artificial Sequence
  Synthetic primer
ccatttgaataagtaacgactaactatg

SEQ ID NO: 5 - DNA, 498 bp, Homo sapiens
agaaagcgatggctaactatgaagcctgatcatttccatgtcatactgagaaagtccccacccttctctgagcctcagtttctctttttataagtaggagtctggagtaaatgatttccaatggctctcatttcaatacaaaatttccgtttattaaatgcatgagcttctgttactccaagactgagaaggaaattgaacctgagactcattgactggcaagatgtccccagaggctctcattcagcaataaaattctcaccttcacccaggcccactgagtgtcagatttgcatgcactagttcacgtgtgtaaaaaggaggatgcttctttcctttgtattctcacatacctttaggaaagaacttagcacccttcccacacagccatcccaataactcatttcagtgactcaacccttgactttataaaagtcttgggcagtatagagcagagattaagagtacagatgctggatccgccctggtagtccacgc

SEQ ID NO: 6 - DNA, 41 bp, Artificial Sequence
  Synthetic primer
agaaagcgatggctaactatgaagcctgatcatttccatgt

SEQ ID NO: 7 - DNA, 37 bp, Artificial Sequence
  Synthetic primer
gcgtggactaccagggaggctccagcatctgtactct

(19) United States
(12) Patent Application Publication
(10) Pub. No.: US 2005/0064451 A1
(43) Pub. Date: Mar. 24, 2005

(54) Methods and compositions for the detection of bacterial species

(75) Inventors: Scott Happe, Austin, TX (US); Justin T. Brown, Manor, TX (US); Frank B. Bozyan, Lubbock, TX (US); Dwight Dubois, Austin, TX (US)

Correspondence Address: PALMER & DODGE, LLP;KATHLEEN M. WILLIAMS / STR, 111 HUNTINGTON AVENUE, BOSTON, MA, 02199, US

(73) Assignee: Stratagene California

(21) Appl. No.: 10/820,971

(22) Filed: Apr. 8, 2004

Related U.S. Application Data
(60) Provisional application No. 60/462,260, filed on Apr. 11, 2003.

(51) Int. Cl. C12Q 1/68; C12P 19/34

(52) U.S. Cl. 435/6; 435/91.2

(57) ABSTRACT

The invention relates to compositions, methods and kits for the PCR-based detection of bacterial species. Methods are provided for increasing the specificity of a PCR-based bacterial assay. More specifically, primer sets and PCR-based assays are provided that amplify and detect conserved 16S rRNA gene sequences from multiple Mycoplasma species. The invention also provides kits for performing those assays, and compositions comprising oligonucleotide primers useful in those assays.

### RELATED APPLICATIONS

The present application claims priority under 35 U.S.C. §119(e) to U.S. Provisional Application with Ser. No. 60/462,260, filed Apr. 11, 2003, the entirety of which is hereby incorporated by reference.

### FIELD OF THE INVENTION

The invention relates to the detection of bacterial species in biological samples.

### BACKGROUND OF THE INVENTION

Various species of Mycoplasma are involved in human and animal pathologies. Mycoplasma contamination of eukaryotic cell cultures is also a common problem, leading to unreliable experimental results and possibly unsafe biological products. These small bacteria pass easily through commonly used 0.22-micron sterilization filters. Antibiotics are often unsuccessful in eradicating the infection due in part to the lack of a Mycoplasma cell wall. Some studies suggest that the prevalence of Mycoplasma contamination in cell cultures is as high as 15% (DelGuidice and Hopps 1978; Barile 1979; McGarrity and Kotani 1985). Such contamination can adversely affect experiments by altering eukaryotic cell surface antigens, chromosomal structure, metabolic rates, protein expression patterns, and transfection efficiency. Detection of these bacteria in cultured cells and tissues is critical for the reliability and reproducibility of experimental data. Traditional methods of detection are difficult due to the fastidious and slow growth conditions of Mycoplasma species in culture (Barile & Razin, 1979, The Mycoplasmas, New York, Academic Press, pp 425-474). Mycoplasma culture tests require 15-30 days and the interpretation of the data requires a trained eye. While staining with 4′,6′-diamidino-2-phenylindole hydrochloride (DAPI) or Hoescht stain reduces turn-around time compared to the culture method, the results can still be difficult to interpret. Immunofluorescence detection is also subjective and insensitive, particularly for Acholeplasma (Tang, Hu et al. 1999).

A number of Mycoplasma detection assays for detection in both clinical and cell culture settings have been described, for example, by: Harasawa et al., 1993, Res. Microbiol., 144: 489-493; Blazek et al., 1990, J. Immunol. Meth. 131: 203-212; Hopert et al., 1993, J. Immunol. Meth. 164: 91-100; McGarrity et al., 1986, In Vitro Cell. Dev. Biol. 22: 301-304; Uphoffet al., 1992, Leukemia 6: 335-341; van Kuppeveld, 1992, Appl. Environ. Microbiol. 58: 2606-2615; van Kuppeveld, 1994, Appl. Environ. Microbiol. 60: 149-152; Wirth et al., 1994, Cytotechnology 16: 67-77; Corless et al., 2000, J. Clin. Microbiol. 38: 1747-1752; Kong et al., 2001, Appl. Environ. Microbiol. 67: 3195-3200; Yoshida et al., 2002, J. Clin. Microbiol. 40: 1451-1455; Loens et al., 2002, J. Clin. Microbiol. 40: 1339-1345; and Eastick et al., 2003, J. Clin. Pathol.: Mol. Pathol. 56: 25-28.

Uphoffet al. (2002, Leukemia 16: 289-293) describe an assay using a mixture of 9 different oligonucleotide primers that amplify 16S rRNA genes from M. arginini, M. fermentans, M. hominis, M. hyorhinis, M. orale, and Acholeplasma laidlawii. Dussurget & Roulland-Dussoix (1994, Appl. Environ. Microbiol. 60: 953-959) describe the use of a mixture of PCR primers that amplify 16S rRNA gene sequences to detect M. arginini, A. laidlawii, M. hyorhinis, M. orale, and M. fermentans.

These references do not teach PCR-based bacterial (e.g., Mycoplasma) detection methods that avoid false positives caused by amplification of E. coli DNA sequences that contaminate preparations of recombinant Taq polymerase. These references also do not teach the simultaneous detection of multiple (e.g., 4 to 8 or more) species of Mycoplasma with four PCR primers or fewer.

### SUMMARY OF THE INVENTION

The invention encompasses methods and compositions that increase the specificity of nucleic acid amplification-based bacterial assays that use recombinant enzymes produced in bacteria, such as E. coli. These methods and compositions take advantage of the recognition that cross-hybridization of amplification assay primers with recombinant host-derived nucleic acids that contaminate preparations of recombinant enzymes is a considerable source of false positive assay results.

The invention also encompasses methods and compositions for the detection of the presence of Mycoplasma in a biological sample. The compositions and methods permit the detection of multiple different species of Mycoplasma with a single set of reagents. The invention further encompasses methods and compositions that increase the specificity of PCR-based bacterial assays. These methods and compositions can reduce the frequency of false positives in PCR-based detection of any non-E. coli bacterial species in which recombinant polymerase is used.

In one aspect, a method is provided for increasing the specificity of a PCR-based bacterial assay, the method comprising: aligning a chosen non-E. coli bacterial target nucleic acid sequence with a homologous E. coli nucleic acid sequence; selecting a PCR primer sequence such that it comprises a sequence perfectly complementary in its three 3′-terminal nucleotides to the chosen non-E. coli bacterial target nucleic acid sequence, and one or more mismatches, in its three 3′-terminal nucleotides, to the homologous E. coli nucleic acid sequence; and performing PCR using the PCR primer sequence in a PCR-based bacterial assay.

In one embodiment, the primer sequence comprises two or more mismatches in its three 3′-terminal nucleotides, relative to the corresponding E. coli nucleic acid sequence.

In another embodiment, the primer sequence comprises three mismatches in its three 3′-terminal nucleotides, relative to the corresponding E. coli nucleic acid sequence.

In another aspect, a composition is provided, comprising an oligonucleotide primer that hybridizes under standard conditions to a nucleic acid sequence comprised by a Mycoplasma 16S rRNA gene, wherein the 3′-terminal two nucleotides of the primer are selected so as not to base pair with template if the oligonucleotide primer cross-hybridizes with an E. coli 16S rRNA gene template.

In one embodiment, the primer hybridizes to a 16S rRNA gene sequence from at least two species of Mycoplasma.

In another embodiment, the primer hybridizes to a 16S rRNA gene sequence from at least three species of Mycoplasma.

In another embodiment, the primer hybridizes to a 16S rRNA gene sequence from at least four species of Mycoplasma.

In another embodiment, the primer hybridizes to a 16S rRNA gene sequence from at least five species of Mycoplasma.

In another embodiment, the primer hybridizes to a 16S rRNA gene sequence from at least six species of Mycoplasma.

In another embodiment, the primer hybridizes to a 16S rRNA gene sequence from at least seven species of Mycoplasma.

In another embodiment, the primer hybridizes to a 16S rRNA gene sequence from at least eight species of Mycoplasma.

In another embodiment, the composition comprises at least two oligonucleotide primers that hybridize to opposite strands of a nucleic acid sequence comprised by a Mycoplasma 16S rRNA gene, wherein the 3′-terminal two nucleotides of at least one of the primers are selected so as not to base pair with template if the at least one oligonucleotide primer cross-hybridizes with an E. coli 16S rRNA gene template.

Another embodiment further comprises an internal amplification control template. In a preferred embodiment, the internal amplification control template has the sequence of SEQ ID NO: 5 or the complement thereof.

In another embodiment, the oligonucleotide primer has a sequence selected from the group consisting of SEQ ID Nos: 1-4.

In another aspect, an assay kit is provided comprising a composition comprising an oligonucleotide primer as described above. In one embodiment, the kit further comprises an internal amplification control template nucleic acid. In another embodiment, the internal amplification control template has the sequence of SEQ ID NO or the complement thereof. In another embodiment, the kit comprises a template-dependent nucleic acid extending enzyme. In another embodiment, the kit comprises a uracil DNA glycosylase enzyme.

In another aspect, an isolated nucleic acid is provided consisting of the sequence of any one of SEQ ID NOs: 1-4 or the respective complement thereof.

In another aspect, an isolated nucleic acid is provided comprising the sequence of SEQ ID NO: (5) or the complement thereof.

In another aspect, a kit is provided comprising an isolated nucleic acid as described above. The kit can further comprise an internal amplification control nucleic acid template. The internal amplification control nucleic acid template can comprise the sequence of SEQ ID NO (5) or the complement thereof. The kit can also comprise a template-dependent nucleic acid extending enzyme and/or a uracil DNA glycosylase enzyme.

In another aspect, a method of detecting the presence of a Mycoplasma species in a sample is provided, the method comprising: forming a reaction mixture comprising the sample and a set of oligonucleotide primers that hybridize under standard conditions to a nucleic acid sequence comprised by a Mycoplasma 16S rRNA gene, wherein the 3′-terminal nucleotide of one or more of the set of primers is selected so as not to base pair with an E. coli 16S rRNA gene template if the oligonucleotide primer cross-hybridizes with the E. coli 16S rRNA gene template; extending the primers; and detecting extension products of the primers, wherein the presence of an extension product is indicative of the presence of a Mycoplasma species in the sample.

In another embodiment of this or another aspect, for at least one member of the set of oligonucleotide primers, the 3′-terminal nucleotide is selected such that it does not base pair with an E. coli 16S rRNA gene template if the member cross-hybridizes with an E. coli 16S rRNA gene template.

In another embodiment of this or another aspect, for at least one member of the set of oligonucleotide primers, the 3′-terminal two nucleotides are selected such that they do not base pair with an E. coli 16S rRNA gene template if the member cross-hybridizes with an E. coli 16S rRNA gene template.

In another embodiment of this or another aspect, the step of extending the primers comprises polymerase chain reaction (PCR) amplification.

In another embodiment of this or another aspect, the reaction mixture further comprises an internal amplification control template, wherein the product of amplification of the internal amplification control template is detectably different in size or sequence than the product of amplification of Mycoplasma 16S rRNA species amplified in the sample. In another embodiment, the internal amplification control comprises a nucleic acid template comprising 5′ and 3′ regions that hybridize with corresponding regions of a 16S rRNA gene sequence from one or more Mycoplasma species under standard conditions, flanking a central region of non-16S rRNA gene sequence, wherein the 5′ and 3′ regions hybridize to oligonucleotide primers in the primer set. In another embodiment, the internal amplification control template comprises the sequence of SEQ ID NO. 5.

In another embodiment of this or another aspect, the method comprises the step, before the step of extending the primers, of contacting the reaction mixture with a uracil DNA glycosylase enzyme.

In another embodiment of this or another aspect, the reaction mixture comprises dUTP.

In another embodiment of this or another aspect, the step of extending the primers is performed in the presence of dUTP, and the extending results in the incorporation of dUTP into an extension product.

In another embodiment of this or another aspect, the step of detecting extension products comprises gel electrophoresis.

In another embodiment of this or another aspect, the set of oligonucleotide primers comprises a primer selected from the group consisting of: (SEQ ID Nos: 1-4).

In another embodiment, the set of oligonucleotide primers comprises SEQ ID Nos: 1 & 2.

In another embodiment, the set of oligonucleotide primers consists of SEQ ID Nos: 1-4.

In another embodiment of this or another aspect, the set of oligonucleotide primers detects the presence of Mycoplasma species including Acholeplasma laidlawii, Mycoplasma arginini, M. fermentans, M. hominis, M. hyorhinis, M. orale, M. salivarium, and M. pirum. Additional species for which detection according to the invention is specifically contemplated include M. hyopneumoniae, M. flocculare, M. hyosynoviae, M. neurolyticum, M. pulmonis, M. pneuminiae, M. capricolum, M. arthritidis, M. gallinarum, M. mycoides, M. bovis, Acholeplasma granularum, A. modicum and A. morum, among others. The specific primer sets described herein (e.g., SEQ ID NOs 1-4) or additional primers prepared using guidance provided herein and knowledge in the art can be applied to arrive at primer sets that recognize these or other Mycoplasma species.

In another aspect, a method is provided for detecting the presence of a Mycoplasma species in a sample, the method comprising: contacting the sample with a set of oligonucleotide primers that hybridize to a 16S rRNA gene sequence in one or more Mycoplasma species, the set of oligonucleotide primers comprising SEQ ID NO: 2; extending at least one of the primers; and detecting an extension product, wherein the presence of an extension product indicates the presence of a Mycoplasma species in the sample.

In one embodiment, the set of oligonucleotide primers further comprises a primer selected from the group consisting of SEQ ID NOs 1, 3 and 4 (mtri1A, mtri1B, mtri1D).

In another embodiment, the set of oligonucleotide primers further comprises oligonucleotide primers of SEQ ID NOs 1 & 3 (mtri1A and mtri1B).

In another embodiment, the set of oligonucleotide primers further comprises oligonucleotide primers of SEQ ID NOs 1 & 4 (mtri1A and mtri1D).

In another embodiment, the set of oligonucleotide primers further comprises oligonucleotide primers of SEQ ID NOs 1, 3 & 4 (mtri1A, mtri1B, and mtri1D).

In another aspect, a method is provided for detecting the presence of a Mycoplasma species in a sample, the method comprising: contacting the sample with a set of oligonucleotide primers that hybridize to a 16S rRNA gene sequence in one or more Mycoplasma species, the set of oligonucleotide primers comprising SEQ ID NO. 1, extending at least one of the primers; and detecting an extension product, wherein the presence of an extension product indicates the presence of a Mycoplasma species in the sample.

In one embodiment, the set of oligonucleotide primers further comprises a primer selected from the group consisting of SEQ ID NOs 2, 3, & 4 (mtri2short, mtri1B, and mtri1D).

In another embodiment, the set of oligonucleotide primers further comprises oligonucleotide primers of SEQ ID NOs 3 & 2 (mtri1B and mtri2short).

In another embodiment, the set of oligonucleotide primers further comprises oligonucleotide primers of SEQ ID NOs 4 & 2 (mtri1D and mtri2short).

In another embodiment, the set of oligonucleotide primers further comprises oligonucleotide primers of SEQ ID NOs 2, 3 & 4 (mtri2short, mtri1B, and mtri1D).

In another aspect, a method is provided for detecting the presence of a Mycoplasma species in a sample, the method comprising: contacting the sample with a set of oligonucleotide primers that hybridize to a 16S rRNA gene sequence in one or more Mycoplasma species, the set of oligonucleotide primers comprising SEQ ID NO. 3; extending at least one of the primers; and detecting an extension product, wherein the presence of an extension product indicates the presence of a Mycoplasma species in the sample.

In one embodiment, the set of oligonucleotide primers further comprises a primer selected from the group consisting of SEQ ID NOs 1, 2 & 4 (mtri1A, mtri2short, and mtri1D).

In another embodiment, the set of oligonucleotide primers further comprises oligonucleotide primers of SEQ ID NOs 1 & 2 (mtri1A and mtri2short).

In another embodiment, set of oligonucleotide primers further comprises oligonucleotide primers of SEQ ID NOs 4 & 2 (mtri1D and mtri2short).

In another embodiment, the set of oligonucleotide primers further comprises oligonucleotide primers of SEQ ID NOs 1, 2 & 4 (mtri1A, mtri2short, and mtri1D).

In another aspect, a method is provided for detecting the presence of a Mycoplasma species in a sample, the method comprising: contacting the sample with a set of oligonucleotide primers that hybridize to a 16S rRNA gene sequence in one or more Mycoplasma species, the set of oligonucleotide primers comprising SEQ ID NO. 4; extending at least one of the primers; and detecting an extension product, wherein the presence of an extension product indicates the presence of a Mycoplasma species in the sample.

In one embodiment, the set of oligonucleotide primers further comprises a primer selected from the group consisting of SEQ ID NOs 1, 2 & 3 (mtri1A, mtri1B, and mtri2short).

In another embodiment, the set of oligonucleotide primers further comprises oligonucleotide primers of SEQ ID NOs 1 & 2 (mtri1A and mtri2short).

In another embodiment, the set of oligonucleotide primers further comprises oligonucleotide primers of SEQ ID NOs 3 & 2 (mtri1B and mtr2short).

In another embodiment, the set of oligonucleotide primers further comprises oligonucleotide primers of SEQ ID NOs 1, 2 & 3 (mtri1A, mtri1B, and mtri2short).

Definitions:

As used herein, the phrase “increasing the specificity” of an assay means reducing the frequency or likelihood of false positive assay results. The specificity of an assay is “increased” relative to another assay if there are at least 10% fewer false positive assay results, and preferably at least 20%, 30%, 50%, 75%, 90% or more, up to and including 100% fewer (no false positives) in that assay relative to the other.

One common source of false positive results in bacterial detection methods based on nucleic acid amplification (e.g., PCR-based assays) is nucleic acid from a recombinant host in recombinant polymerase preparations.

As used herein, the term “PCR-based bacterial assay” refers to an assay method for the detection or quantitation of a given bacterial genus or species in a sample, in which the assay comprises PCR amplification with two or more primers that amplify one or more nucleic acid sequences from the targeted bacterial genus or species. A “PCR-based bacterial assay” as the term is used herein is not intended or designed to detect the presence or amount of E. Coli bacteria in a sample.

As used herein, the term “aligning” when used in reference to nucleic acid sequences means arranging one or more sequences relative to another such that the greatest number of identical nucleotides are aligned with each other. BCM Search Launcher (via hypertext transfer protocol at //searchlauncher.bcm.tmc.edu/), formatted with BOXSHADE 3.2.1 on the Swiss EMBnet node server (available via hypertext transfer protocol on the world wide web at ch.embnet.org/software/BOX_form.html) can be used for primer sequence alignments. Multiple sequence alignments can also be performed using the BLAST suite of programs available from the NCBI website (see below).

As used herein, the term “homologous” means evolutionarily related. A host bacterial nucleic acid sequence (e.g., an E. coli nucleic acid sequence) is “homologous” to a bacterial target nucleic acid sequence (or vice versa) if it is at least 50% identical to the bacterial target sequence. For the purposes of the present invention, a homologous nucleic acid sequence of a recombinant host bacterium (e.g., E. coli) includes the known sequence or sequences in the recombinant host's genome which has (have) the highest homology to a selected target gene sequence in a target bacterial species. In many instances, homology will be well known, for example, the 16S rRNA gene sequences of Mycoplasma sp. are well known to be homologous to the 16S rRNA gene sequence from E. coli (e.g., the M. orale 16S rRNA gene sequence is a known homolog and is 81% identical to the E. coli 16S rRNA gene sequence). Homology between a target species nucleic acid sequence and a recombinant host (e.g., E. coli) nucleic acid sequence is determined by sequence alignment using, for example, Basic BLAST (e.g., Version 2.0, Altschul et al., 1997, Nucleic Acids Res. 25: 3389-3402) set with default parameters (descriptions default=500; alignments default=100; expect=10; filter=off; matrix=BLOSUM62). When two known sequences are to be aligned, the “Blast 2 Sequences” program can be used to align and determine homology (bl2seq; Tatusova & Madden, 1999, FEMS Microbiol. Lett. 174: 247-250). The “Blast 2 Sequences” program, available through the NCBI website can be used with default alignment parameters. This program produces the alignment of two given sequences using the BLAST engine for local alignment. Default parameters (for use with the BLASTN program only) are as follows: Reward for a match: 1; Penalty for a mismatch: −2; Strand option Both strands; open gap penalty 5; extension gap penalty 2; gap x_dropoff 50; expect 10.0; word size 11; and Filter (checked).

As used herein, the term “hybridizes,” when used in reference to an oligonucleotide primer, refers to the formation of a hydrogen-bonded base paired duplex with a nucleic acid having a sequence sufficiently complementary to that of the oligonucleotide primer to permit the formation of such a duplex. As the term is used herein, exact complementarity between an oligonucleotide primer and a nucleic acid sequence is not required, with mismatches permitted as long as the resulting duplex is a substrate for extension by a template-dependent nucleic acid extending enzyme. A nucleic acid sequence is “sufficiently complementary” to an oligonucleotide primer if the primer can form a duplex with a molecule comprising the nucleic acid sequence at 55° C. that can be extended by at least one nucleotide by a template-dependent nucleic acid extending enzyme, e.g., a polymerase, in a solution comprising 10 mM Tris-HCl, pH 8.8, 50 mM KCl, 2.0 mM MgCl₂ and 200 μM each of dATP, dCTP, dGTP and dTTP.

As used herein, the phrase “standard conditions,” when used in reference to nucleic acid hybridization refers to incubation at 55° C. in a buffer containing 10 mM Tris-HCl, pH 8.8, 50 mM KCl, and 2.0 mM MgCl₂. Oligonucleotide primer molecules hybridized to a template nucleic acid (e.g., a Mycoplasma 16S rRNA gene or an internal amplification control template) under these conditions will be extended by at least one nucleotide by a template-dependent nucleic acid extending enzyme provided that the 3′-terminal two nucleotides of the primer are base paired to the template.

As used herein, the phrase “Mycoplasma species” is intended to encompass members of the genus Mycoplasma and members of the genus Acholeplasma.

As used herein, the phrase “Mycoplasma 16S rRNA gene” refers to 16S rRNA gene sequences from members of the genus Mycoplasma and from members of the genus Acholeplasma.

As used herein, the phrase “cross-hybridizes” refers to the hybridization of an oligonucleotide primer designed to hybridize with a Mycoplasma species 16S rRNA gene sequence with a 16S rRNA from a non-Mycoplasma species.

As used herein, the phrase “does not base pair with” or “is mismatched” means that a given sequence of nucleotides on an oligonucleotide primer does not form complementary hydrogen bonds with an adjacent nucleotide sequence on a nucleic acid molecule. As the phrase is used herein, when one or more 3′-terminal nucleotides on an oligonucleotide primer “do not base pair” with a template nucleic acid molecule, a template-dependent nucleic acid extending enzyme will not extend the primer by one nucleotide or more under annealing and polymerization conditions as follows: 10 μCi of each of ³³P-labeled dATP, dCTP, dGTP and dTTP (>1000 Ci/mMole), 1× Taq polymerase buffer (10 mM Tris-HCl, pH 8.8, 50 mM KCl, 1.5 mM MgCl₂, 0.001% (w/v) gelatin), 100 nM of primer, 2.0 mM MgCl₂, 100 fmol template and 0.04 U/μl of Taq2000™ polymerase (Stratagene #600197-51); the mixture is heated at 94° C. for 30 seconds, annealing is performed at 55° C. for 30 seconds, and polymerization is performed at 72° C. for one minute. The presence of one or more labeled species detected by autoradiography when the reaction products are separated on polyacrylamide gel demonstrates the extension of the primer. If there are no labeled species, the terminal nucleotide(s) of the primer “does not base pair with” the template. Alternatively, when the sequence of a potential contaminating template, e.g., an E. coli 16S rRNA gene sequence, is known, one can manually or via computer (e.g., using BLAST, with default parameters) align a given primer sequence with the contaminating template sequence. If one or more (e.g., one, two, three) of the 3′-terminal three nucleotides of the primer are not complementary to the template, they “do not base pair” with the template.

As used herein, the phrase “internal amplification control template” refers to a double- or single-stranded nucleic acid molecule that is added to a nucleic acid amplification reaction to serve as a control for the activity of the template-dependent nucleic acid extending enzyme used in such reaction. An internal amplification control template useful according to the methods disclosed herein is amplified using members of the same oligonucleotide primer set used to amplify Mycoplasma 16S rRNA gene sequences, yet differs in size and sequence from targeted Mycoplasma 16S rRNA gene sequences.

As used herein, the phrase “template-dependent nucleic acid extending enzyme” refers to an enzyme that catalyzes the template-dependent addition of nucleotides to the 3′ end of a nucleic acid strand hybridized to a substantially complementary template nucleic acid strand. A template-dependent nucleic acid extending enzyme useful in the methods disclosed herein will not extend an oligonucleotide primer in which one or more 3′-terminal nucleotides (e.g., the last 3′-terminal nucleotide, the last two 3′-terminal nucleotides, etc.) is not base paired with the template nucleic acid. That is, a template-dependent nucleic acid extending enzyme useful in the methods disclosed herein requires that the 3′ terminal two nucleotides of the primer strand be base paired with the template. Base pairing of the 3′-terminal two nucleotides of a primer with the template can be determined by alignment of the sequences, either manually or by computer—if the last one or two 3′ nucleotides of the primer are complementary to the template, the template-dependent nucleic acid extending enzyme useful in the methods described herein will extend the primer by at least one nucleotide, and preferably more under conditions as described in the definition of “does not base pair,” above. If, on the other hand, the alignment shows that the last one or two nucleotides are not complementary to the template, a template-dependent nucleic acid extending enzyme useful in the methods described herein will not extend the primer by one or more nucleotides under the same conditions.

As used herein, the phrase “uracil DNA glycosylase enzyme” refers to a DNA repair enzyme that catalyzes the hydrolysis of uracil residues from single-stranded or double-stranded DNA. The removal of uracil residues from a DNA molecule leaves an abasic site rendering the DNA strand subject to cleavage by heat under alkaline conditions and non-functional as a template for amplification. Uracil DNA glycosylase enzymes, e.g., E. coli Uracil DNA glycosylase, are commercially available.

As used herein, the term “isolated” refers to a population of molecules, e.g., polypeptides, polynucleotides, or oligonucleotides, the composition of which is less than 50% (by weight), preferably less than 40% and most preferably 2% or less, contaminating molecules of an unlike nature.

As used herein, the term “set” refers to a group of at least two. Thus, a “set” of oligonucleotide primers comprises at least two oligonucleotide primers.

As used herein, the phrase “extension product” refers to the nucleic acid product of an extension reaction catalyzed by a template-dependent nucleic acid extending enzyme. An “extension product” has been extended by at least one nucleotide by a template-dependent nucleic acid extending enzyme.

As used herein, the phrase “detectably different in size or sequence” means that the extension or amplification product formed by enzymatic extension or amplification of an internal amplification control template can be distinguished from the extension or amplification product of a target nucleic acid on the basis of a difference in size or sequence. Conditions are well known for the separation of nucleic acids differing by as little as one nucleotide in length. Thus, the phrase “detectably different in size or sequence” means that a molecule differs by at least one nucleotide in length from another. It is preferred, however, that molecules of “detectably different” size differ by more than one nucleotide, e.g., by at least 10 nucleotides, 50 nucleotides, 100 nucleotides or more. Alternatively, molecules of different sequence can be distinguished, e.g., on the basis of an enzymatic cleavage site or a binding site for a ligand that is present on one nucleic acid molecule but not on the other. Such molecules are thus of “detectably different” sequence.

### BRIEF DESCRIPTION OF THE FIGURES

FIG. 1 shows a diagram of an Internal Amplification Control (IAC) template. The 5′ moieties of primers nic1A and nic1B (grey portions of crooked arrows) are identical to primers mtri1A and mtri2short (straight grey arrows), respectively. They are thus homologous to a region of the Mycoplasma 16S rRNA gene (dark grey shading). The 3′ moieties of primers nic1A and nic2B (black portions of crooked arrows) are homologous to regions of the human β-globin gene (light grey shading). Using human genomic DNA as a template, the resulting amplification product can be used as an internal amplification control template (SEQ ID NO: 5). Additionally, the experimental mtri1A/mtri2short primer set amplifies both the IAC and a region of the 16S rRNA gene.

FIG. 2 shows the results of experiments demonstrating that the Mycoplasma 16S primer set disclosed herein detects 10⁵ copies of genomic DNA from eight Mycoplasma species of interest. Amplification was performed using the Brilliant® Master Mix (Stratagene), the 16S primer set mtri1A, mtri1B, mtri1D, mtri2short (SEQ ID NOs: 1-4), and 10⁵ copies of genomic DNA from each of the species indicated as the most common cell culture contaminants (Tang, Hu et al. 1999) (lanes 1-8), or water as a no-template control (lanes 9 and 10). Mor=M. orale, Ala=A. laidlawii, Mar=M. arginini, Msa=M. salivarium, Mpi=M. pirum, Mfe=M. fermentans, Mho=M. hominis, Mhy=M. hyorhinis, NTC=no-template control. Note that bands are not observed in the NTC lanes, suggestive of a lack of detection of residual E. coli DNA in the SureStart® Taq polymerase. Far left lane, kB marker.

FIG. 3 shows the results of experiments examining whether the Mycoplasma 16S primer set mtri1A, mtri1B, mtri1D, mtri2short (SEQ ID NOs: 1-4) amplifies a product from human or mouse genomic DNA templates. Amplification was performed using the Brilliant® Master Mix with either the Mycoplasma 16S (100 nm), mouse β-actin (200 nM, Stratagene #302110-14), or human 13-actin (200 nM, Stratagene #302010-14) primer sets. Each primer set was mixed with genomic DNA from either Mycoplasma orale (1000 input copies), mouse (1.6×10⁶ copies, Big Blue), human (#1193-1), all three species, or no DNA (no-template control, NTC). Genomic DNA was obtained from the Stratagene Production group at BioCrest. The Mycoplasma 16S primer set does not amplify a product from either human or mouse GDNA templates, indicating that it will not produce false-positive results due to the presence of DNA from cultured cells. Note that the control β-actin primers cross-react between human and mouse species. Far left lane, kB maker.

FIG. 4 shows the results of experiments investigating the effect of intentional primer 3′ mismatch with corresponding 16S rRNA gene of E. coli. Amplification was performed using the Brilliant® Master Mix and either the 16S primer set (“3′ Mismatched Primers”) or a second primer set (“3′ Matched Primers”). The forward primer of the “3′ Matched Primer” set is shifted downstream by ten bases with respect to the mtri1A primer, such that the last ten 3′ terminal bases exactly match the E. coli sequence (see Table 2). The forward primer sequence is: 5′-GCCTAACTACTATGTGCCAGCAGC-3′; the reverse primer sequence is: 5′-GCGTGGACTACCAGGGTATCT-3′. Each primer set was used to amplify template from M. orale gDNA (1000 copies), E. coli gDNA (1000 copies), or no template. The 3′ Matched Primers detect E. coli and produce a false-positive in the no-template control reaction, whereas the 16S Mismatched Primers (mtri1A, mtri1B, mtri1D) do not detect E. coli and shows a clean no-template control. Left lane, kB marker.

FIG. 5 shows the results of experiments evaluating the Internal Amplification Control template. (A) Amplification was performed using human genomic DNA and the nic1A/nic1B primer set (SEQ ID NOs 6 and 7) in the absence of dUTP. The reaction producing the product observed in the right lane was performed in the presence of 1.3 μg of human genomic DNA. The no-template control reaction did not show an amplification product (data not shown). Left lane, kB marker. (B) Amplification was performed using 250 copies of internal amplification control template per 50 μl PCR reaction, the indicated input copy number of genomic DNA template from M orale, and the 16S primer set (mtri1A, mtri1B, mtri1D/mtri2short, SEQ ID NOs: 1-4). Left lane, kB marker.

FIG. 6 shows a comparison of results obtained with the commercially available ATCC Mycoplasma Detection Kit versus results obtained using the mtri1A, mtri1B, mtri1D/mtri2short (SEQ ID NOs: 1-4) primer set. M. orale and A. laidlawii genomic DNA were titrated from 1000 copies down to 10 copies and amplified with the Brilliant® Master Mix containing the 16S primer set mtri1A, mtri1B, mtri1D/mtri2short (SEQ ID NOs: 1-4) and 250 copies of IAC. The same gDNA dilutions were used with the ATCC Mycoplasma Detection Kit (Version 2.0) and amplified according to the manufacturer's recommendations. Both the 16S primer set disclosed herein and the ATCC primers amplify from at least 10 copies of input M. orale GDNA, while the ATCC kit is capable of amplifying 5-fold less A. laidlawii GDNA. The ATCC kit produces template concentration-independent results due to the nested PCR protocol. The nested PCR protocol produces higher product yield and greater overall sensitivity, but also requires two thermal cycling steps and is prone to the formation of non-specific products.

FIG. 7 shows the results of experiments investigating the use of uracil DNA glycosylase to reduce carryover contamination. The 16S primer set mtri1A, mtri1B, mtri1D/mtri2short (SEQ ID NOs: 1-4) amplifies Mycoplasma gDNA in the presence of UDG and efficiently eliminates amplification of uracil-containing PCR products. 1000 copies of M. orale gDNA were used as a template with the 16S primer set to amplify the 315-bp Mycoplasma product in a reaction containing dUTP. The band was excised from the gel and purified with the StrataPrep™ gel extraction kit. 10⁴ copies of M. orale gDNA or the indicated amounts of the purified uracil-containing amplicon were used as templates in a reaction containing the Brilliant Master Mix and the 16S primer set. The reactions were incubated at 37° C. for 10 minutes, the UDG was inactivated at 94° C. for 10 minutes, and the reactions were cycled according to the standard protocol. When the uracil-containing DNA was used as a template, amplification was observed only in the absence of UDG. UDG does not affect the amplification of the gDNA control. Kb ladder was used as a marker.

FIG. 8 shows the results of experiments investigating the optimal MgCl₂ concentration in Mycoplasma 16S rRNA gene sequence amplification reactions with Taq polymerase. The PCR was performed using Taq2000 polymerase (Stratagene) and the associated Taq buffer containing 1.5 mM MgCl₂ (final). Additional MgCl₂ (25 mM stock, Roche) was added from 0 to 1.25 mM (1.5 mM to 2.75 mM, final). The samples were prepared in duplicate. At 1.5 mM MgCl₂, little product is observed. Product yield increases to a maximum at 2.25 mM MgCl₂, but small (<250 bp) non-specific products increase from 2.25 to 2.75 mM MgCl₂. Based upon repeated experiments, 2.0 mM MgCl₂ gave the most consistent results with the smallest amount of non-specific products.

FIG. 9 shows the results of experiments evaluating the ability of the 16S Mycoplasma primer set mtri1A, mtri1B, mtri1D, mtri2short (SEQ ID NOs: 1-4) to detect Mycoplasma DNA from a contaminated cell culture. A HeLa cell culture of unknown infection status was obtained from the Production group at BioCrest (Cedar Creek, Tex.). The results indicate that the cell culture is contaminated with Mycoplasma, as verified by both the 16S and Mycoplasma Plus™ primer sets. Neither medium alone (minimal essential media, ATCC), nor medium containing 10% horse serum (ATCC), resulted in an amplification product with either primer sets. Kb ladder was used as a marker as indicated.

### DETAILED DESCRIPTION OF THE INVENTION

In one aspect, methods are provided for increasing the specificity of PCR-based bacterial assays. More specifically, methods are provided that reduce the frequency of false positive results in PCR-based bacterial detection assays that use recombinant polymerase. It is recognized herein that a common source of false positive results in PCR-based bacterial assays that use recombinant polymerase is that preparations of recombinant polymerase are most often contaminated with genomic DNA from the host bacterium, which is usually, but not necessarily, E. coli. Where the host bacterium, e.g., E. coli, has a homologous sequence to the target gene sequence in the bacterial genus or species being detected, the use of recombinant preparations of, e.g., Taq polymerase will result in false positive amplification results when the primers cross-hybridize and permit extension from contaminating host species (e.g., E. coli) genomic DNA template.

Thus, methods and compositions are provided herein in which PCR-based bacterial assays are given increased specificity (i.e., lower false positive rates) by selecting PCR primer sequences for the assays such that primers that may cross hybridize with contaminating template nucleic acid from the recombinant polymerase host specices will not be extended and will not result in amplification products.

Primer Design:

To design primers that exclude the amplification of contaminating host nucleic acid present in recombinant polymerase preparations, the chosen nucleic acid sequence for a target bacterial species (or genus, e.g., a group of Mycoplasma or other bacterial species) is aligned with a homologous nucleic acid sequence from the species used as host in the production of the polymerase to be used in the PCR-based bacterial assay. From the aligned sequences, a primer is selected such that it is perfectly complementary in its three 3′-terminal nucleotides to the chosen bacterial target nucleic acid sequence or sequences, but has one or more, preferably two or more, mismatches in its three 3′ terminal nucleotides, relative to the homologous sequence from the recombinant polymerase host bacterium. Primers for this and other aspects of the invention should be at least 12 nucleotides in length, and preferably 15-25 nucleotides in length, but can be longer, e.g., 30, 35, 40, 45 or 50 nucleotides or more in length, but are generally 100 nucleotides or less. For illustration, examples of mismatches in the 3′-terminal three nucleotide positions are described below:

| | |
|---|---|
| 5′------------------XXX 3′ | (primer) |
| 5′------------------XXC 3′ | (primer) |
| 5′------------------XAX 3′ | (primer) |
| 5′------------------GXX 3′ | (primer) |
| 5′------------------XAC 3′ | (primer) |
| 5′------------------GXC 3′ | (primer) |
| 5′------------------GAX 3′ | (primer) |

| | |
|---|---|
| 5′₋₋₋₋₋₋₋₋₋₋₋₋₋₋₋₋₋₋GAC₋₋₋₋₋₋₋₋₋₋₋₋₋₋₋₋₋3′ | (complement) |
| 3′₋₋₋₋₋₋₋₋₋₋₋₋₋₋₋₋₋₋CTG₋₋₋₋₋₋₋₋₋₋₋₋₋₋₋₋₋5′ | (host template) |

(“X” is any of G, A, T or C that is not complementary to the template strand)

Of course, further mismatches with the recombinant polymerase host bacterial template sequence are also permitted, as long as there is at least one mismatch in the 3′ terminal three nucleotides of the primer.

Sequence Alignment:

The alignment of a target bacterial nucleic acid sequence (e.g., a 16S rRNA sequence from a Mycoplasma strain) with a homologous sequence from a recombinant host bacterium, e.g., E. coli, can be performed by one of skill in the art by computer using software that is widely available. For example, where a homologous sequence is already known, the “Blast 2 Sequences” program (bl2seq; Tatusova & Madden, 1999, FEMS Microbiol. Lett. 174: 247-250) can be used. The program is available through the NCBI website and can be used with default alignment parameters. This program produces the alignment of two given sequences using the BLAST engine for local alignment. Default parameters (for use with the BLASTN program only) are as follows: Reward for a match: 1; Penalty for a mismatch: −2; Strand option Both strands; open gap penalty 5; extension gap penalty 2; gap x_dropoff 50; expect 10.0; word size 11; and Filter (checked).

Where homologs are not known, or where one, for example, wishes to determine whether there are homologs with a higher degree of homology than a known homolog, BLAST alignment can be performed against nucleic acid sequences from the recombinant host species. For example, the genome sequence of the recombinant host can be searched and similar sequences aligned. For this purpose, a BLAST alignment can be preformed using the BLASTN program of the Basic BLAST suite of programs (Basic BLAST, Version 2.0, Altschul et al., 1997, Nucleic Acids Res. 25: 3389-3402) set with default parameters (descriptions=500; alignments=100; expect=10; filter=off; matrix=BLOSUM62).

Genome sequences are known for a number of bacterial strains important to human health and to industry. In particular, the E. coli K12 genomic sequence is available at GenBank Accession No. U00096. A large number of other bacterial genome sequences are available on the TIGR Microbial Genome database (available on the World Wide Web at tigr.org/tdb/mdb/mdbcomplete.html), such as the genomes for B. subtilis (Kunst et al., 1997, Nature 390: 249-256), Methanobacterium thermoautotrophicum (Smith et al., 1997, J. Bacteriol. 179: 7135-7155, and Pseudomonas putida (Nelson et al., 2002, J. Environ. Microbiol. 4: 799-808), to name a few. One of skill in the art can therefore determine the presence of sequences homologous to a chosen target bacterial sequence by BLAST analysis when one of the organisms in this database (or in another source, for that matter) is used as a host to produce a recombinant enzyme used in a PCR-based bacterial assay.

PCR Analysis:

The selected primer sequence is then used in a PCR-based assay (in conjunction with one or more additional primers that can optionally have one or more 3′ mismatches with host bacterial template sequence) to amplify the bacterial target nucleic acid sequence. Due to the mismatch(es) with recombinant host template nucleic acid sequence at the 3′ end of the selected primer sequence, primers cross-hybridized to contaminating recombinant host nucleic acid will not be extended by polymerase enzyme, and an amplification product will not be produced from that template. Using this method, the frequency of false-positive results caused by contaminating nucleic acid from preparations of recombinant polymerase will be reduced. The presence of an amplified band of an expected size detected after gel electrophoresis of PCR amplification products confirms the presence of the target bacterium or genus of target bacteria. Other detection approaches, e.g., the use of molecular beacons, are specifically contemplated. Controls should include a “no template” negative control, in which primers, buffer, enzyme(s) and other necessary reagents (e.g., MgCl₂, nucleotides) are cycled in the absence of added test sample. A positive control including a known target template should also be run in parallel.

It is also noted that this method can be used whenever a recombinant enzyme produced in bacteria, including a non-polymerase recombinant enzyme, is used in a mixture that is ultimately subjected to a PCR amplification of a target gene sequence from a different bacterial species. Thus, if, for example, a recombinant uracil DNA glycosylase or other recombinant enzyme is used in treatment or pre-treatment of a sample to be subjected to amplification, this approach will avoid false positive signal from recombinant host nucleic acid introduced with that recombinant enzyme.

PCR-based bacterial detection assays are well known in the art, and rely upon the ability of a set of primers specific for a given gene or nucleic acid sequence (or set of such sequences sharing common primer hybridization sequences) to direct the amplification of a target bacterial sequence from among a background of non-target sequences. Target bacterial genes are often selected to vary as widely as possible from other known sequences in order to ensure the specificity of the assay. However, particularly where one designs an assay to detect more than one species of a given genus, e.g., to detect multiple members of the genus Mycoplasma, it can be necessary to select target sequences that are well conserved among the target genus, e.g., 16S rRNA gene sequences. Because such target sequences also tend to be conserved throughout bacterial species in general, the approach in which primers are designed not to be extension substrates when cross-hybridized to non-target species templates that may be present in recombinant enzyme preparations provides a valuable advantage.

Numerous different PCR protocols are known in the art and exemplified herein below and can be directly applied or adapted for use in the presently-described methods. The specific design of one or more of the necessary primers to avoid extension of primers cross-hybridized to contaminating nucleic acid template from recombinant host bacteria used to prepare one or more of the recombinant enzymes can reduce or eliminate false positive results caused by such contaminating template.

It is further contemplated that bacterial assays that use other types of enzyme-mediated amplification, for example 3SR (Self-Sustained Sequence Replication; Gingeras et al., 1990, Annales de Biologie Clinique, 48(7): 498-501; Guatelli et al., 1990, Proc. Natl. Acad. Sci. U.S.A., 87: 1874), or SDA (Strand Displacement Amplification; Walker, 1994, Nucleic Acids Res. 22: 2670-7), can also benefit from the disclosed methods where the enzymes are recombinantly produced. The presence of contaminating recombinant host nucleic acid will pose the same false-positive problems in any such system that is dependent upon the extension of a hybridized primer for its signal and for its specificity.

The method described above can be applied to assays for the detection of any species of bacteria, in, for example, clinical, research or industrial settings. It is of particular interest in assays designed to detect bacterial pathogens, such as species of Aeromonas, Actinomyces, Bacillus, Bacteriodes, Bordetellas, Borellia, Brucella, Campylobacter, Citrobacter, Clostriduim, Enterobacter, Klebsiella, Proteus, Salmonella, Serratia, Shigella, Yersinia, Enterococcus, Haemophilus, Helicobacter, Listeria, Micrococcus, Neisseria, Pseudomonas, Staphylococcus, Streptococcus, and Vibrio, among others.

Mycoplasma Detection:

In another aspect, compositions and methods are provided for the detection of multiple Mycoplasma species in biological samples. More specifically, assays are provided based upon the PCR amplification of conserved 16S rRNA gene sequences from multiple Mycoplasma species, including the most common species that contaminate cell cultures, and primer sets useful in those assays. Because the bacterial 16S rRNA is not shared by eukaryotic ribosomes, the 16S rRNA provides a suitable amplification target for the detection of Mycoplasma bacteria in eukaryotic cell culture samples or in clinical samples. The invention also provides kits for performing those assays, and compositions comprising oligonucleotide primers as disclosed.

Mycoplasma Detection Assay:

In one aspect, a PCR-based Mycoplasma detection assay capable of detecting the presence of multiple different Mycoplasma species in a single assay. In one embodiment, the assays detect eight or more species of Mycoplasma in a reaction with four or fewer PCR primers that hybridize to and direct the amplification of sequences from the 16S rRNA gene of Mycoplasma species. Assays can be carried out essentially as follows:
1) Prepare a 50 μl mixture of dNTPs (typically 200 μM each dATP, dCTP, dGTP, dTTP; dTTP can optionally be substituted, for example with 400 μM dUTP), enzyme buffer (e.g., 1× Taq polymerase buffer (e.g., 10 mM Tris-HCl, pH 8.8, 50 mM KCl, 1.5 mM MgCl₂, 0.001% (w/v) gelatin), 100 nM of each primer (necessarily a pair including a “forward” or “upstream” primer and a “reverse” or “downstream” primer, but preferably a set of 4 primers, 3 forward and one reverse (for example SEQ ID NOs: 1-4) (see below)), 2 mM MgCl₂, and extending enzyme, e.g., 0.04 U/μl of Taq2000™ polymerase (Stratagene #600197-51);
2) Add 5 μl of a sample to be tested for the presence of Mycoplasma DNA, and cycle for 35 cycles of 30 sec. at 94° C., 1 min. at 55° C., and 1 min at 72° C.;
3) Detect amplified product, e.g., by gel electrophoresis and ethidium bromide staining or equivalent method. Incorporation of one or more labeled nucleotides can permit detection by, for example, fluorescence.

The presence of an amplified product (preferably, but not necessarily a single amplified band) indicates the presence of a Mycoplasma species in the source from which the sample was taken.

A no-sample negative control should be run in parallel, and all assays (including the negative control assay) will preferably comprise an internal control template nucleic acid (see below) that generates a distinctly sized amplicon from what is expected for the experimental templates.

Variations on the exact amounts of the various reagents and on the conditions for the PCR (e.g., buffer conditions, cycling times, etc.) that lead to similar amplification or detection results are known to those of skill in the art and are considered to be equivalents.

Eight species of Mycoplasma account for greater than 95% of cell culture contamination, including Acholeplasma laidlawii, Mycoplasma arginini, M. fermentans, M. hominis, M. hyorhinis, M. orale, M. pirum, and M. salivarium (Tang, et al., 1999, In Vitro Cell Dev. Biol. 35: 1-3). Acholeplasma laidlawii and M. pirum tend to be more difficult to detect than the other common species because they differ more widely from the other species. The assays described herein detect the presence of at least these eight species in a single assay. Further features of the assays disclosed herein include: 1) consistent amplification of genomic DNA (gDNA) from target Mycoplasma species, with as little as 10-100 copies of GDNA per PCR reaction; 2) the reagents and methods described permit, where desired, the production of a single PCR product of the same size for all species of interest; 3) elimination of false positive assay results caused by the presence of E. coli nucleic acid in preparations of recombinant enzymes used to amplify target gene sequences; 4) prevention of carry-over contamination from previous assays; and 5) a robust, well characterized internal amplification control template to control for the presence of inhibitors of the amplification reaction.

The highly conserved nature of the 16S rRNA gene sequences makes it possible to design small sets of primers (e.g., 2, 3 or 4 members) that recognize multiple (e.g., 2, 3, 4, 5, 6, 7, 8 or more) Mycoplasma species.

Primer Design:

For design of a new primer set for the detection of Mycoplasma species, attention was focused on genes for which sequence data was known for a majority of the species of interest. The genomic 16S rRNA gene sequences are available from GenBank via the web site of the National Center for Biotechnology Information (via hypertext transfer protocol on the world wide web at ncbi.nlm.nih.gov/Genbank/) for eight of the most common Mycoplasma species that infect cell cultures: Acholeplasma laidlawii (NCBI ID#M23932), Mycoplasma arginini (NCBI ID#M24579), M. fermentans (NCBI ID#M24289), M. hominis (NCBI ID#M24473), M hyorhinis (NCBI ID#M24658), M. orale (NCBI ID#M24659), M. pirum (NCBI ID#M23940), M. salivarium (NCBI ID#M24661), and E. coli (NCBI ID#2367315). Additional Mycoplasma 16S rRNA gene sequences are also available through GenBank.

The sequences for the 16S rRNA genes from the eight species noted above were aligned along with that of the E. coli 16S region (see Table 2). BLAST analyses were performed remotely through programs on the web site of the National Center for Biotechnology Information (via hypertext transfer protocol on the world wide web at ncbi.nlm.nih.gov/BLAST/) using default parameters, unless otherwise noted. The multiple sequence alignment seen in Table 2 was performed using the BCM Search Launcher (via hypertext transfer protocol at //searchlauncher.bcm.tmc.edu/) and formatted with BOXSHADE 3.2.1 on the Swiss EMBnet node server (via hypertext transfer protocol on the world wide web at ch.embnet.org/software/BOX_form.html).

Following alignment, primer sequences are selected representing regions that are highly conserved between Mycoplasma species. For example, the “mtri1A” forward primer sequence 5′-AGAAAGCGATGGCTAACTATG-3′ (SEQ ID NO: 1) is perfectly conserved between M. salivarium, M. arginini, M. orale, and M. hominis, and is conserved at the 3′ end with regard to Acholeplasma laidlawii, M. fermentans, M. pirum, and M. hyorhinis (see Table 2). As another example, the “mtri2short” reverse primer sequence 5′-GCGTGGACTACCAGGG-3′ (SEQ ID NO: 2) is perfectly conserved between M. salivarium, M. arginini, M. orale, M. fermentans, M. hominis, M. hyorhinis, and Acholeplasma laidlawii, and differs at only two internal nucleotides from the corresponding sequence in the M. pirum 16S rRNA gene sequence.

The alignment with E. coli 16S rRNA sequence was performed in order to identify regions of Mycoplasma 16S rRNA gene sequence that are not well conserved in the E. coli 16S rRNA gene sequences. The extreme 3′ end of the forward primer (mtri1A) is designed to contain a 2-base mismatch with the corresponding sequence of the K12 E. coli 16S rRNA gene. This design prevents the amplification of E. coli gDNA templates that sometimes contaminate preparations of recombinant template-dependent nucleic acid extending enzymes, such as preparations of recombinant Taq polymerase (Corless et al., 2000, J. Clin. Microbiol. 38: 1747-1752), thereby avoiding a source of false positive results.

In one aspect then, primers were designed (denoted “mtri1A” (SEQ ID NO: 1, upstream primer) and “mtri2short” (SEQ ID NO: 2, downstream primer)) that amplify a region of 16S rRNA gene sequence from multiple different Mycoplasma species. The expected PCR product size for Mycoplasma 16S rRNA species amplified using the mtri1A and mtri2short primer set is 315 bp. In this aspect, then, a primer set for the detection of multiple Mycoplasma species comprises the primers having the sequences of mtri1A (SEQ ID NO: 1) and mtri2short (SEQ ID NO: 2).

Due to small differences among species in the 16S rRNA gene sequence recognized by the mtri1A forward primer, two additional forward primers were designed for inclusion in a larger primer set: “mtri1B” (SEQ ID NO: 3) and “mtri1D” (SEQ ID NO: 4). These primers are more specific for A. laidlawii and M. pirum respectively, the two most divergent species in terms of the 16S sequence, and also contain the intentionally designed two nucleotide mismatch with E. coli 16S rRNA at the 3′ terminus.

In another aspect, then, a primer set that detects the presence of at least eight species of Mycoplasma thus includes primers having the sequences of mtri1A (SEQ ID NO: 1), mtri1B (SEQ ID NO: 3), mtri1D (SEQ ID NO: 4) and mtri2short (SEQ ID NO: 2).

In order to avoid false positives, primers selected for the amplification of Mycoplasma 16S rRNA gene fragments should also be examined for the possible recognition of known eukaryotic, e.g., mammalian genome sequences. To do this, BLAST analyses are performed against human and mouse genomes at a decreased stringency level (1000-fold increase over default expect value). Useful primers will have no significantly related mouse or human genomic sequences.

Primers designed following the parameters described above are shown in Table 1. Primer syntheses for the Examples herein below were carried out by Integrated DNA Technologies (Coralville, Iowa). All primers were produced using cyanoethyl phosphoramidite chemistry, ammonium hydroxide deprotection, and desalting by gel filtration chromatography. Results of the PCR were similar regardless of further purification of the primers by high performance liquid chromatography (HPLC) or polyacrylamide gel electrophoresis (PAGE).

Biological Sample Preparation

For testing of the methods described herein, Mycoplasma bacterial strains were obtained as lyophilized preparations from the American Type Culture Collection (ATCC) as follows: Acholeplasma laidlawii (ATCC#23206), Mycoplasma orale (ATCC#29802), M. hominis (ATCC#23114), M. fermentans (ATCC#19989), M. hyorhinis (ATCC#17981), M. pirum (ATCC#25960), M. salivarium (ATCC#23064), M. arginini (ATCC#23243), and E. coli (ATCC#10798).

Crude DNA isolation for sample testing is performed by resuspending lyophilized bacterial cells in 1 ml of sterile double-deionized (ddI) water, followed by thorough mixing with an aerosol-filtered pipette tip. Resuspension in water ensures hydrolysis of the cells and release of the bacterial DNA. The cell solution can be stored at −20° C. or immediately purified with, for example the DNA Stat™ Blood Kit (Stratagene, catalog #400760). Using that kit, 200 μl of sample is applied to the microspin cup included with the kit. 600 μl of WBC Lysis Solution is added and incubated for 5 minutes. The sample is centrifuged for 2 minutes at maximal speed and washed twice with 600 μl each of 1× Wash Solution. The spin cup is dried with a final spin. 200 μl of Elution Buffer is added, and the sample is incubated for 5 minutes and eluted from the column. Genomic DNA samples are stored at −20° C. DNA samples are routinely diluted in either ddI water or 5 mM Tris, pH 7.5, 0.1 mM EDTA (5T.1E) before use as a template in amplification reactions.

When samples are from eukaryotic cell culture, the following sample preparation procedure can be followed. Add 100 μl of cell culture supernatant to a microcentrifuge tube, tightly seal the top and boil (or heat to 95° C., e.g., in a temperature block) for 5 minutes. Spin briefly in a microcentrifuge to move condensation to the bottom of the tube. Add 10 μl of a preparation of hydroxylated silica particles (e.g., StrataClean™ resin) and mix. Pellet the resin by centrifugation and remove the supernatant to a fresh tube. The sample is ready for analysis for Mycoplasma contamination, and is stable for several days at 4° C. Phenol:Chloroform extraction and ethanol precipitation can be used as an alternative.

As another alternative, a protocol can be used that provides cell-equivalent standardization and a more sensitive detection limit for cell lines whose growth is inhibited by Mycoplasma. Such a procedure is as follows. First, harvest adherent cells with trypsin using standard techniques. Take one ml (greater than 100,000 cells) and spin in a microcentrifuge 10-15 seconds to pellet. Wash the pelleted cells twice with sterile phosphate buffered saline, then resuspend in 1 ml of PBS and count cells under a microscope. Aliquot 100,000 cells to a microcentrifuge tube, pellet and remove the supernatant. Resuspend the cells in 100 μl of sterile UV-irradiated water. Boil the cell suspension for 10 minutes and spin briefly to remove condensation. Mix 10 μl of a preparation of hydroxylated silica particles with the suspension as above, and pellet the resin. Take the supernatant to a fresh tube. Five μl of the resulting supernatant is equivalent to extract from 5,000 cells. Dilutions representing 50 and 5,000 cell equivalents should be tested using the methods described herein. Strong Mycoplasma infections are detected in as little as 10 cell equivalents, while weak or cell-growth-inhibiting infections can require cell equivalents in the 500 to 5,000 cell range.

Samples from, for example, clinical sources can also be prepared by boiling and purifying with resin as described above, or for example, by phenol:chloroform extraction and DNA precipitation.

Amplification

The methods and considerations necessary for PCR amplification are well known to those of skill in the art. Exemplary conditions are as follows.

Amplification reactions can be performed in a total volume of 50 μl using a thermal cycler. Reactions are assembled on ice and typically allow for 5 μl of template. The final recipe after assay optimization includes: 200 μM each of dATP, dCTP, and dGTP (Amersham #27-2035-01), and 400 μM of dUTP (added to avoid carryover contamination, see below; Amersham #27-2040-01), 1× Taq polymerase buffer (10 mM Tris-HCl, pH 8.8, 50 mM KCl, 1.5 mM MgCl₂, 0.001% (w/v) gelatin), 100 nM of each primer (e.g., mtri1A, mtri1B, mtri1D, and mtri2short), 0.5 mM additional MgCl₂ (for a final concentration of 2.0 mM), 0.04 U/μl of Taq2000™ polymerase (Stratagene #600197-51), and, if included, 0.02 U/μl UDG (again, to eliminate carryover contamination, see below; New England Biolabs, # M0280S). Double-distilled ionized water is used in lieu of template samples in no-template controls.

Alternatively, Brilliant® QPCR Master Mix (Stratagene, #600549-51) can be used in place of Taq polymerase buffer, Taq2000™, and dNTPs/dUTP. Extra magnesium need not be added when using the Brilliant® Master Mix. The components of the Brilliant® Master Mix (2×) are: 60 mM Tris-HCl, pH 8.0, 40 mM KCl, 11 mM MgCl₂, 400 μM each dATP/dCTP/dGTP, 800 μM dUTP, 0.02% Triton X-100, 0.02% Tween-20, 12% glycerol, and 0.05U/μl SureStart® Taq polymerase.

In one aspect, the assay mixture is as follows:

| | | Final Assay | |
|---|---|---|---|
| Component | Stock Solutions | Concentrations | Volume |
| 10X buffer | 10X | 1X | 5 μl |
| dNTP/dUTP mix | 10 mM dATP, dCTP, dGTP | 200 μM dATP, dCTP, dGTP | 1 μl |
| | 20 mM dUTP | 400 μM dUTP | |
| Magnesium | 25 mM | 2 mM | 1 μl^{#} |
| chloride* | | | |
| Primer mix | 2.5 μM each primer | 100 nM | 2 μl |
| Internal | 125 copies/μl | 5 copies/μl | 2 μl |
| amplification | (62.5 ag/μl) | (2.5 ag/μl) | |
| control template* | | | |
| deionized water | — | — | 33.6 μl |
| Taq DNA | 5 U/μl | 0.04 U/μl | 0.4 μl |
| polymerase | | | |
| Template/Extract | — | — | 5 μl^{$} |
| | | Final: | = 50 μl |

^{#}Assuming the 10X buffer provides 1.5 mM magnesium chloride final; if different, adjust accordingly.
*See description below.
^{$}User may add more template, if desired, although the risk of PCR inhibition increases with template volume
Note that archeal DNA polymerase (e.g. Pfu) are not recommended for amplification in assays using dUTP because they tend to be poisoned by dUTP.

The final optimized thermal cycler conditions are as follows: 35 cycles of 30 sec (denaturation), 1 min (annealing), and 1 min (extension). Cycling time is about 1.5 hours. If UDG is included, the reactions are preincubated at 37° C. for 10 minutes for optimal hydrolysis of dUTP-containing DNA followed by 94° C. for 10 minutes to inactivate UDG. For PCR mixtures containing the Brilliant® Master Mix or hot-start PCR enzymes, a 10-minute preincubation is included to activate the SureStart® polymerase/hot-start component.

Internal Amplification Control Template

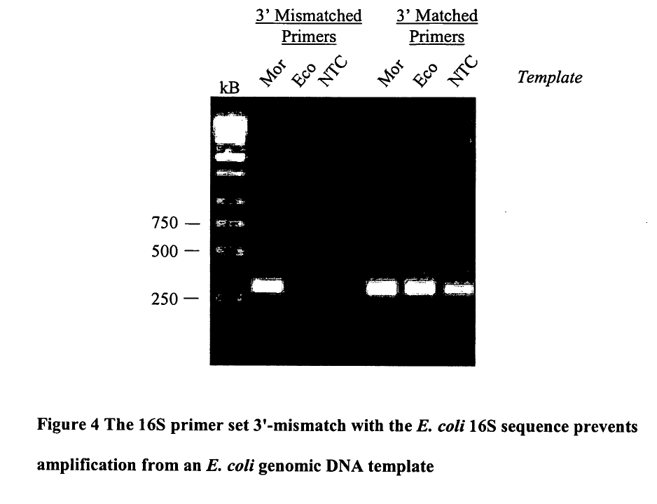

Internal amplification controls are essential to diagnostic tests as gauges of assay inhibition (DuBois, D. B. et al., 1999, Standards for PCR Assays in PCR Applications, Academic Press). Residual media components and cellular debris from cell cultures could potentially inhibit Taq DNA polymerase and produce false-negative results. The internal amplification control template is designed to be amplified by the same primers that amplify the target diagnostic sequence. The ideal internal amplification control (IAC) for an assay that utilizes gel-based analysis should produce a band whose molecular weight is easily discernable from the experimental band. For this assay, an IAC was chosen that would produce a band of size of about 500 bp (Illustration 1), in comparison to the 315 bp Mycoplasma PCR product. Additionally, this experimental design circumvents any problems associated with multiplex PCR, where two separate primer sets are used in one reaction. In this design, one set of primers amplifies both native Mycoplasma 16S rDNA (“target band”) and a 458-base pair region of the human β-globin gene cloned between the Mycoplasma primer binding sites for mtri1A and mtri2short (an additional 37 bases). When amplifying the human β-globin gene with primers flanked by the Mycoplasma target priming sites, a PCR product of the expected size (495 bp) was successfully amplified (FIG. 4, panel A and Table 1). This PCR product was purified and served as the IAC template. Any non-16S rRNA sequence can be used as the central part of an IAC template, although it is preferred that the sequence have approximately the same G+C content as the targeted Mycoplasma 16S rRNA gene sequences.

Reduction of Carryover From Previous Assays

One problem encountered when laboratories routinely use the same primer sets for assays such as the Mycoplasma detection assays described herein is that small amounts of the amplified products from previous assays can contaminate subsequent reactions, giving false positive results. To avoid this problem, the Mycoplasma detection assays described herein can be routinely carried out in the presence of dUTP, which permits the user to eliminate carry-over PCR products with uracil DNA-glycosylase (UDG). In the event that amplification product is inadvertently carried over from one experiment to another, the enzyme UDG will catalyze the release of free uracil found in the contaminating product and hydrolysis of the DNA strand. A preincubation of the PCR reaction for 10 minutes at 37° C. activates the UDG enzyme. Heat energy in a subsequent denaturation step (10 minutes at 94° C.) eliminates the UDG activity, activates the DNA polymerase (if modified for “hot-start” activation, e.g. SureStart® Taq), and catalyzes the cleavage of the contaminating abasic phosphodiester backbone. UDG is commercially available, e.g., from New England Biolabs (Cat. # M0280S).

Kits

On one aspect, a kit is provided containing reagents and instructions necessary to perform the Mycoplasma detection assays described herein. In a specific aspect, then, the kit can comprise a set of two or more, and preferably four primers as described herein that recognize and amplify a 16S rRNA gene sequence from a group of Mycoplasma species.

In additional aspects, the kit can further comprise an IAC template, a positive control (e.g., Mycoplasma genomic DNA), a template-dependent nucleic acid extending enzyme (preferably a thermostable template-dependent nucleic acid extending enzyme, such as Taq polymerase), a necessary buffer, additional reagents needed by the enzyme, such as MgCl₂, dNTPs, dUTP and/or a UDG enzyme.

### EXAMPLES

### Example 1

### Detection of Eight Different Species of Mycoplasma with One Primer Set

The primer set described in Table 1 was tested for the detection of each of eight different species of Mycoplasma: Acholeplasma laidlawii, Mycoplasma arginini, M. fermentans, M. hominis, M. hyorhinis, M. orale, M. pirum, and M. salivarium. Crude DNA isolation was performed by resuspending lyophilized Mycoplasma bacterial cells in 1 ml of sterile double-deionized (ddI) water, followed by thorough mixing with a aerosol-filtered pipette tip. Resuspension in water ensured hydrolysis of the cells and release of the bacterial DNA. The cell solution was stored at −20° C. or immediately purified with a DNA Stat™ Blood Kit (Stratagene, catalog #400760): 200 μl of sample was applied to the microspin cup included with the kit. 600 μl of WBC Lysis Solution was added and incubated for 5 minutes. The sample was centrifuged for 2 minutes at maximal speed and washed twice with 600 μl each of 1× Wash Solution. The spin cup was dried with a final spin. 200 μl of Elution Buffer was added, and the sample was incubated for 5 minutes and eluted from the column.

Amplification reactions were performed in a total volume of 50 μl using a RoboCycler thermal cycler (Stratagene) or single-block Techne thermal cyclers (models Genius or Flexigene). Reactions were assembled on ice and typically allowed for 5 μl of template. The final recipe after assay optimization included: 200 μM each of dATP, dCTP, and dGTP (Amersham #27-2035-01), and 400 μM of dUTP (Amersham #27-2040-01), 1× Taq polymerase buffer (10 mM Tris-HCl, pH 8.8, 50 mM KCl, 1.5 mM MgCl₂, 0.001% (w/v) gelatin, Stratagene #600131-82), 100 nM of each primer (mtri1A, mtri1B, mtri1D, and mtri2short), 0.5 mM additional MgCl₂ (for a final concentration of 2.0 mM), 0.04 U/μl of Taq2000™ polymerase (Stratagene #600197-51), and, if included, 0.02 U/μl UDG (New England Biolabs, # M0280S). Double-distilled ionized water was used in lieu of template samples in no-template controls. Alternatively, the Brilliant® QPCR Master Mix (Stratagene, #600549-51) was used in place of Taq polymerase buffer, Taq2000™, and dNTPs/dUTP. Extra magnesium was not added when using the Brilliant® Master Mix. The components of the Brilliant® Master Mix (2×) are: 60 mM Tris-HCl, pH 8.0, 40 mM KCl, 11 mM MgCl₂, 400 μM each dATP/dCTP/dGTP, 800 μM dUTP, 0.02% Triton X-100, 0.02% Tween-20, 12% glycerol, and 0.05U/μl SureStart® Taq polymerase. Note that archaeal DNA polymerases (e.g. Pfu) are not recommended for amplification in this assay due to poisoning by dUTP.

The final optimized thermal cycler conditions are as follows: 35 cycles of 30 sec (denaturation), 1 min (annealing), and 1 min (extension). If UDG is included, the reactions were preincubated at 37° C. for 10 minutes for optimal hydrolysis of dUTP-containing DNA followed by 94° C. for 10 minutes to inactivate UDG. For PCR mixtures containing the Brilliant® Master Mix or hot-start PCR enzymes, a 10-minute preincubation was included to activate the SureStart®D polymerase/hot-start component.

For gel electrophoresis, 10-μl samples of 50-μl amplification reactions (unless otherwise noted) were fractionated on 2% agarose gels containing 0.125 μg/ml ethidium bromide (SeaKem LE, BioWhittaker Molecular Applications #50004). While 2% gels produce the greatest resolution between IAC and target bands, 1% gels produce acceptable results (data not shown). Gels were run at 120V in 1× TAE for about 55 minutes. Kb molecular weight marker (Stratagene #201115) was used to estimate molecular weight. Gels were imaged using the Eagle Eye II Still Video System (Stratagene). Alternative means of detection are contemplated, e.g., real-time PCR detection with fluorescent label.

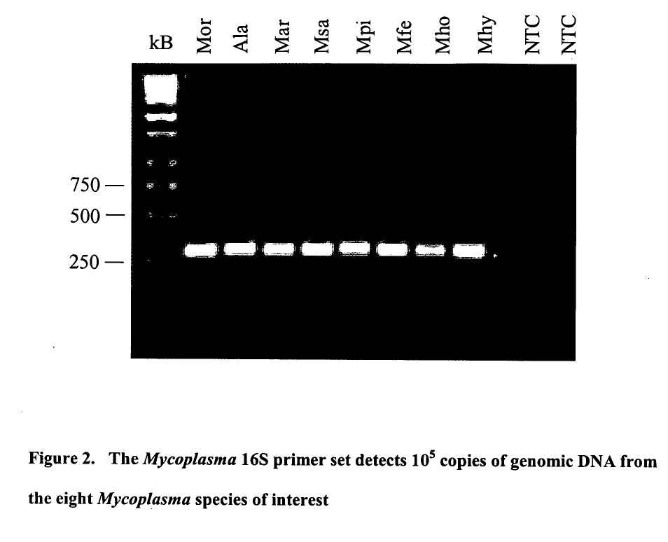

Using crude DNA preparations from the eight species of lyophilized Mycoplasma and Acholeplasma cells as templates, the 16S primer set (mtri1A (SEQ ID NO: 1, mtri1B (SEQ ID NO: 3), mtri1 (SEQ ID NO: 4) and mtri2short (SEQ ID NO: 2)) recognizes all 8 species (FIG. 2). The amount of DNA tested in the initial PCR (10⁵ copies) is comparable to the amount of Mycoplasma DNA that would be found in a contaminated culture (10⁷-10⁸ cells/ml of culture, 5 μl of extracted supernatant added to the PCR=5×10⁴−5×10⁵ input copies) (McGarrity, G. J. and H. Kotani, 1985, The Mycoplasmas. Barile & Razin. New York, Academic Press: pp 353-390).

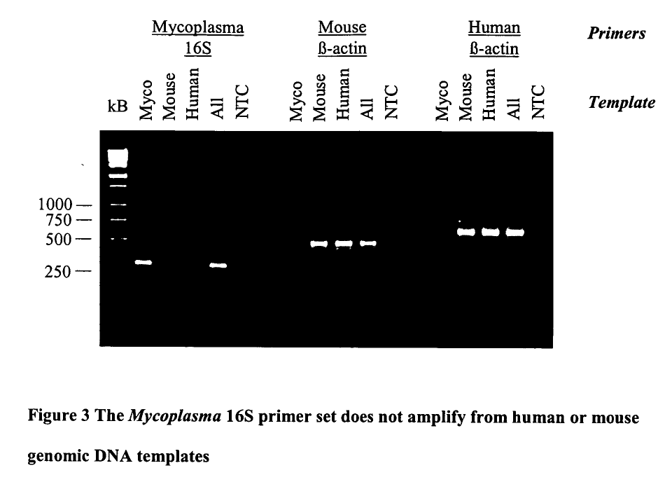

It was also investigated whether the primers were likely to hybridize with eukaryotic sequences, as this assay was designed to test potentially infected eukaryotic cell cultures. BLAST analyses were performed against human and mouse genomes at a decreased stringency level (1000-fold increase over default expect value). No significantly related sequences were returned (data not shown). Consistently, no unexpected products have been observed when amplification has been performed in the presence of mouse or human genomic DNAs (FIG. 3).

### Example 2

### Elimination of E. coli Reactivity Reduces False Positives

Throughout the early development of a 16S primer set to detect Mycoplasmas, it was determined that the initially-designed primer sets frequently showed false-positive results in the absence of genomic DNA template. Due to the similarity between the E. coli 16S region and that of Mycoplasmas (see Table 2), it was reasoned that these primers may be detecting small amounts of contaminating E. coli DNA present in recombinant Taq polymerase preparations. Therefore, the Mycoplasma 16S primers (mtri1A/mtri1B/mtri1D/mtri2short) were designed to eliminate detection of E. coli. As stated previously, all primers in the optimized 16S primer set contain a mismatch at the 3′ end with the E. coli sequence (see Table 2 and Table 1). Such a design should prevent extension of the primers from E. coli templates with Taq DNA polymerase.

The prevention of E. coli 16S rRNA gene amplification was examined by comparing the 16S primer set to a second primer set that exactly matched the E. coli sequence at the 3′ end (mtri1/mtri2) (FIG. 4). The mismatched primer set robustly detects M. orale genomic DNA, but does not detect E. coli genomic DNA. The matched primer set, however, detects both M. orale and E. coli with similar sensitivity, in addition to showing a strong false-positive band in the no-template control. These results demonstrate that the mismatched primer set disclosed herein is less likely to show false-positive results due to E. coli DNA contamination.

### Example 3

### Synthesis and Testing of an Internal Amplification Control Template

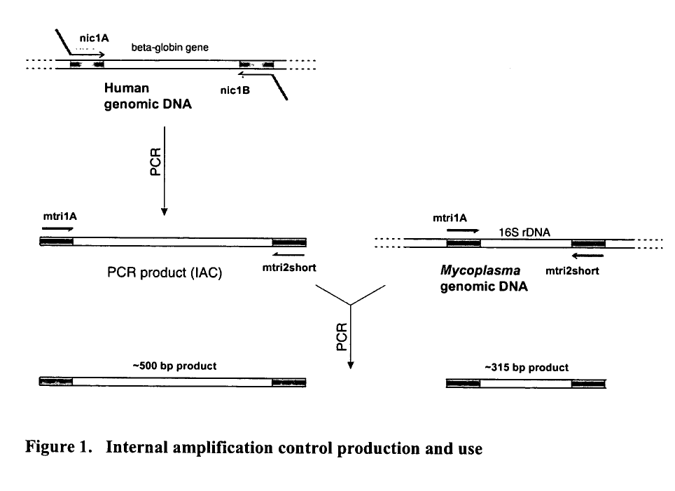

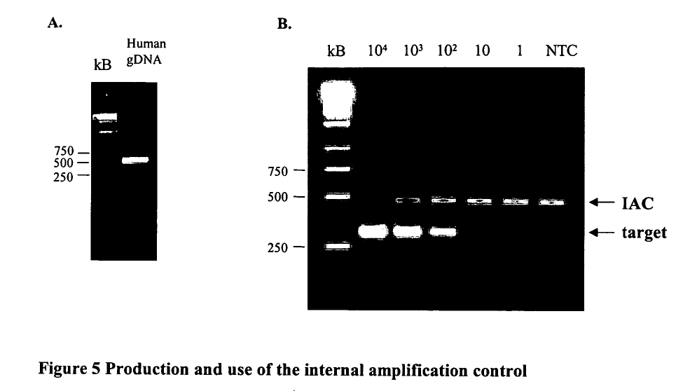

The Internal Amplification Control template shown in Table 1 was tested in amplification reactions with and without Mycoplasma genomic DNA. The IAC was designed to be amplified by the same primers as the target Mycoplasma amplification product (FIG. 1). Forward primer nic1A (SEQ ID NO: 6) and reverse primer nic1B (SEQ ID NO: 7) (Table 1) contained 5′ moieties that are homologous to the target region of Mycoplasma 16S rDNA and 3′ moieties that are homologous to a region of the human β-globin gene (NCBI gi:18266749). The %(G+C) nucleotide content of the amplified region of the Mycoplasma genome (43 to 49%, depending on species) was similar to that of the amplified region of the human genome (43%). The 495 bp IAC was produced by Taq2000™ amplification in a reaction containing 200 μM each of dATP, dCTP, dGTP, and dTTP (Stratagene), 0.04 U/μl of UDG (New England BioLabs), 200 μM of the primer set nic1A/nic1B, and 1.3 μg of human genomic DNA. The thermal cycler protocol was: 37° C. for 10 min, 94° C. for 10 min, 35 cycles of (94° C. for 30 sec, 55° C. for 1 min, and 72° C. for 1 min), followed by a single cycle of 72° C. for 5 min. The IAC product (see FIG. 5) was purified using the StrataPrep PCR Purification Kit (Stratagene) and quantified using a Beckman spectrophotometer. The PCR product was diluted in steps of 1:10 in 5 mM Tris/0.1 mM EDTA to a working concentration of 62.5 ag/μl, with no less than 10 μl of each subsequent dilution to minimize loss of DNA. Tween-20 (Sigma, 0.1% final) was added in later preparations of IAC to minimize non-specific binding and improve consistency of amplification.

To demonstrate amplification of the IAC template by the 16S primer set in the presence of genomic DNA, amplification was performed on an empirically optimized amount of IAC template (250 copies per 50 μl PCR reaction) with variable input gDNA copy number. The data in FIG. 5, Panel B, demonstrate that the IAC amplified as expected with primer set mtri1A/mtri1B/mtri1D/mtri2short, even in the presence of Mycoplasma GDNA. At gDNA concentrations of >500-1000 copies, the IAC signal begins to decline. The sensitivity of the assay is not altered by the low amount of internal amplification control template included in the reaction (data not shown).

### Example 4

### Analysis of the Sensitivity of Mycoplasma Detection Assays

As described (FIG. 2), a 16S primer set as disclosed herein successfully amplified 10⁵ input copies of genomic DNA from eight target Mycoplasma species. At greater than 1000 input copies (typical Mycoplasma infections are greater than 107 cfu/ml of culture, McGarrity, G. J. and H. Kotani, 1985), the 16S primer set produced sharp, robust bands, with few non-specific products.

Next, the performance of the assay at low DNA copy numbers was examined. Based upon rough estimates of the size of Mycoplasma genomes (Neimark & Lange, 1990, Nucl. Acids Res. 18: 5443-5448), the concentrations of the crude genomic DNA preparations for each species were calculated in copies/μl and normalized for each species. The DNAs were titrated into reactions containing the 16S primer mix. Faint bands from the reactions with the lowest template concentration that were not apparent in the no template control samples (NTC) demonstrate that this assay can yield observable product from as few as one to ten input copies of Mycoplasma orale DNA (FIG. 5, Panel B).

A comparison of DNA titrations between the eight species of Mycoplasma examined (Table 3) revealed that the sensitivity was greatest for M. orale (reproducibly detected with as few as 1 copy), M. fermentans, and M. arginini, and least for A. laidlawii, the most divergent species in terms of sequence of the 16S rRNA gene. The inclusion of the mtri1B forward primer improved the detection limit for A. laidlawii from 500-1000 copies to 50-100 copies (data not shown).

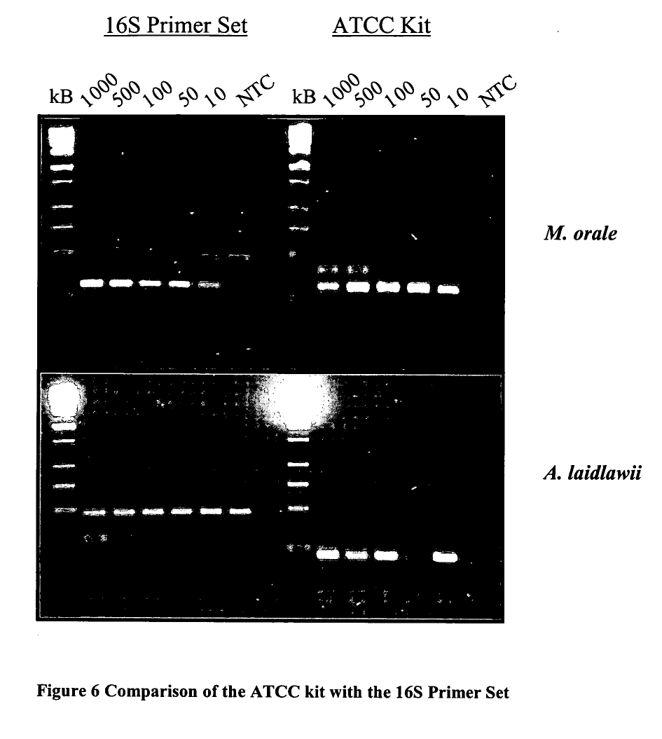

The results of assays using the primers described herein were compared with the results of assays using the commercially available Mycoplasma Detection Kit from ATCC. The ATCC kit uses primers complementary to the 16S-23S rRNA gene spacer region in a two-staged, nested PCR format. In this format, the products of the first PCR are used with a second set of primers in a second PCR amplification. To compare the results obtained with the ATCC kit with those obtained using the methods described herein, first-stage primers were used to amplify gDNA from M. orale or A. laidlawii using SureStart® Taq polymerase (Stratagene) and the ATCC polymerase buffer included with the kit. For no-template controls, ddI water replaced gDNA. The thermal cycling protocol was 2 minutes at 94° C. followed by 30 cycles of 94° C. for 30 sec, 55° C. for 30 sec, and 72° C. for 1 min. 5 μl of the products of the first-stage reaction were added to a second tube containing the second-stage primers, SureStart® Taq polymerase, and ATCC polymerase buffer. The second-stage PCR cycling conditions were identical to the first-stage. The results demonstrated that both the 16S primer set mtri1A/mtri1B/mtri1D/mtri2short (SEQ ID NOs: 1-4) and the ATCC nested PCR primers detect M. orale with similar sensitivities (FIG. 6, top panel), but the ATCC assay detected A. laidlawii (FIG. 6, bottom panel) at a 5- to 10-fold lower concentration (10 copies for ATCC vs. 50-100 copies for the 16S primer set). This higher sensitivity may be due to the nested format of the ATCC kit, as it re-amplifies from a PCR product template in the second stage. However, nested assays require longer cycling times, allow more room for user error, and show a propensity for non-specific products. In addition, the ATCC assay produces multiple bands that differ for each species of Mycoplasma (FIG. 6), and the product intensity is not template-concentration dependent. In a preferred aspect, the assays disclosed herein are designed as “yes” or “no” diagnostic tests producing a single band for ease of interpretation.

### Example 5

### Reduction of Potential Assay Contamination Using UDG

The application of the uracil DNA glycosylase method of reducing or eliminating carryover contamination of PCR product from previous assays was examined by performing assays in the presence of dUTP. Amplified uracil-containing product was spiked into subsequent reactions that were treated with UDG (0.02 U/μl UDG; New England Biolabs) for 10 minutes at 37° C. before PCR was initiated. UDG was inactivated by incubation at 94° C. for 10 minutes before routine PCR cycling.

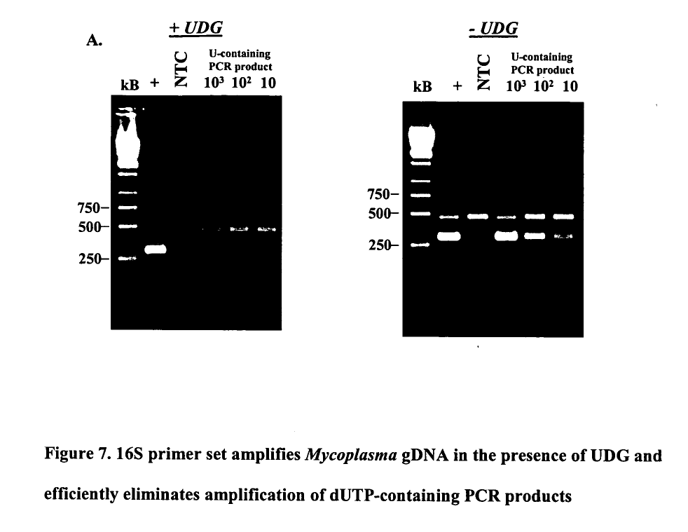

The data in FIG. 7 demonstrate that UDG treatment inhibits amplification of uracil-containing PCR product—even at amplicon concentrations that are orders of magnitude higher than typically expected for carryover contamination. Neither the presence of UDG, nor the two 10-minute preincubations, reduce apparent yield of PCR product in reactions containing 10⁴ copies of M. orale gDNA (FIG. 7). The amplification of Mycoplasma gDNA is reduced only slightly at very low copy numbers (<500 copies, data not shown). Internal amplification control product is sometimes reduced slightly in the presence of UDG (FIG. 6, compare the two panels).

### Example 6

### Examination of the Effect of Magnesium Concentration

The specificity and product yield was found to vary based upon the amount of magnesium chloride present in the reaction. Initially, the assay was developed using Stratagene's Brilliant® QPCR Master Mix, containing 5.5 mM magnesium chloride (final assay concentration) and SureStart® Taq polymerase (Stratagene). While the Brilliant® Master Mix was developed specifically for quantitative PCR using the MX-4000 real-time instrument, it is also useful as a general, validated PCR reagent with optimal dNTP/dUTP ratio for carry-over prevention. When using the Brilliant® Master Mix format, the PCR was specific (only target and IAC bands were observed on the gel). In addition, product yields were consistently highest using the Brilliant® Master Mix.

In order to investigate the use of other reaction mix formulations in the Mycoplasma assays disclosed herein, assays were performed using additional polymerase and reaction mix preparations. In particular, it was sought to be determined whether the inclusion of dUTP affected results when using other reaction mix and enzyme formulations. Use of Taq2000™ DNA polymerase (Stratagene) with a standard dNTP mix (200 μM each) without dUTP produced results similar to the Brilliant® Master Mix format, without the need for additives beyond those found in the associated Taq polymerase buffer.

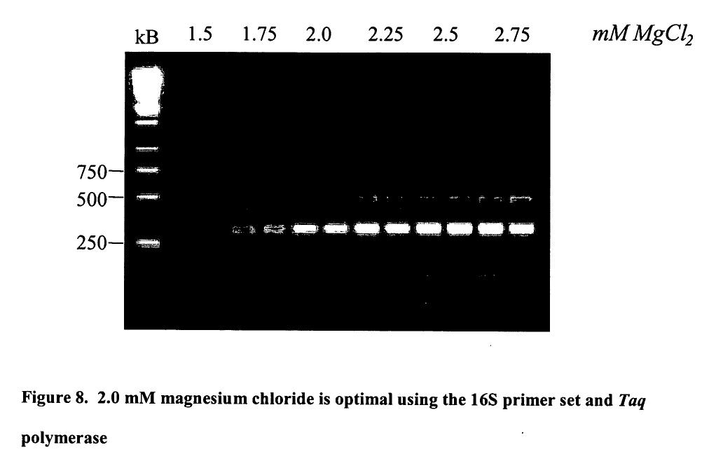

However, upon amplification with Taq2000™, Taq buffer, and 200 μM dNTPs (lacking dTTP)/400 μM dUTP, little or no product was formed. It was reasoned that additional magnesium may be required due to the higher concentration of dUTP in the reaction. Consequently, increasing the magnesium chloride concentration dramatically improved results with Taq2000 when dUTP was present (FIG. 8). 2.0 mM magnesium chloride was found to be optimal, whereas typical polymerase buffers contain 1.5 mM MgCl₂. Concentrations of magnesium higher than 2.0 mM reduced the stringency of the assay, as several smaller, non-specific PCR products were also observed. SureStart® Taq (Stratagene), and two other Taq polymerase preparations (AmpliTaq Gold™ hot-start polymerase (Applied Biosystems) and Invitrogen's recombinant Taq polymerase) were also found to require 2.0 mM magnesium for optimal product yield with dUTP (data not shown).

Therefore, it is recommended that when performing the assays as disclosed herein in the presence of dUTP and with a Taq polymerase, that the PCR be conducted in a buffer containing a final concentration of 2.0 mM MgCl₂. Other polymerases/buffers that have not been tested may vary slightly in the absolute amount of magnesium required for optimal amplification with the 16S primers. Differences in thermal cyclers, water and sample purity, and other factors may alter assay performance as well with respect to magnesium concentration. Therefore, it may be necessary for the user to optimize the magnesium concentration. Given the guidance provided herein, such optimization is well within the abilities of one of skill in the art.

### Example 7

### Analysis of HeLa Cell Cultures for Mycoplasma Contamination

To determine the utility of the 16S primer set mtri1A/mtri1B/mtri1D/mtri2short (SEQ ID NOs: 1-4) for Mycoplasma testing of cell cultures, a HeLa cell culture was obtained. Prior to testing, the Mycoplasma contamination status of the cell culture was unknown. First, serial dilutions of M. orale gDNA were spiked into HeLa cell culture samples to determine if Mycoplasma gDNA could be detected in a culture that may be negative for Mycoplasma cells. As negative controls, medium alone was tested, with or without horse serum. Cell culture samples were prepared as follows: 1 ml of harvested cells was washed twice in phosphate-buffered saline (PBS), and resuspended in a final volume of 1 ml PBS. The cells were counted, and 50,000 cell equivalents were resuspended in 100 μl of sterile water. For samples containing spiked DNA, the indicated copy number of M. orale DNA was added to the resuspended cells before washing and counting. The cells (or media alone for the negative control samples) were boiled for 10 minutes, and 10 μl of StrataClean™ resin was added to the extract. After a final brief spin, the supernatant (clarified extract) was transferred to a new tube and 5 μl was used directly in a PCR assay with the 16S primer set mtri1A/mtri1B/mtri1D/mtri2short (SEQ ID NOs: 1-4) and the internal amplification control template of SEQ ID NO: 5. This primer set was compared with that in the Mycoplasma Plus™ kitcurrently sold by Stratagene.

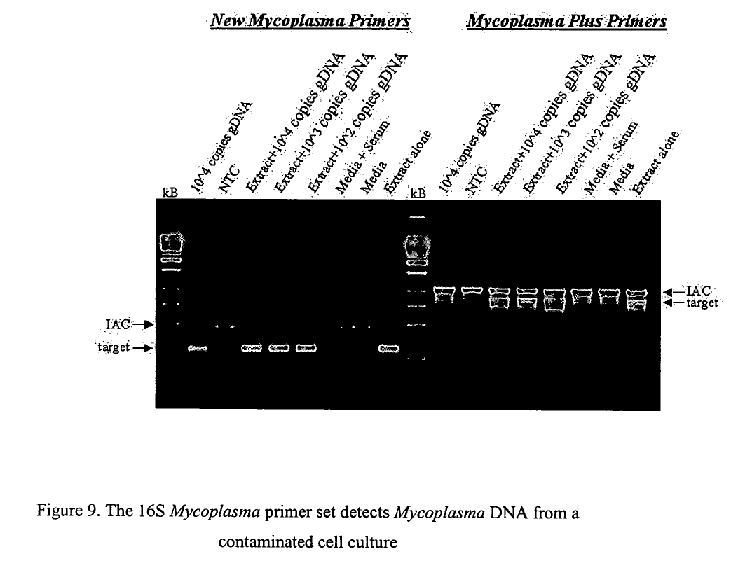

The data in FIG. 9 demonstrate that the cell culture was in fact contaminated with Mycoplasma. Both the Mycoplasma Plus™ primer set and the 16S primer set mtri1A/mtri1B/mtri1D/mtri2short (SEQ ID NOs: 1-4) produce a target band of the appropriate size for the respective assay (FIG. 9, “Extract alone”). Medium and medium containing horse serum do not produce target product (FIG. 9, “Media+Serum” and “Media”), indicating that the contamination results from the cell line and not from the reagents in which the cells were cultured. Note that the addition of purified Mycoplasma gDNA to the cultures does not increase the target band intensity (FIG. 9, “Extract+N copies gDNA”), suggesting that the amount of Mycoplasma gDNA in the HeLa culture is at or greater than the upper detection limit of the assays. As 10⁴ copies of GDNA produces a band of lower intensity than the extract itself, the infection of this culture may be at or greater than 10⁴ Mycoplasma cells per 50,000 HeLa cells. These data demonstrate that the 16S primer set mtri1A/mtri1B/mtri1D/mtri2short (SEQ ID NOs: 1-4) is capable of detecting Mycoplasma both with purified gDNAs and with cultures contaminated with Mycoplasma cells. Since the Mycoplasma Plus™ primer set also produced a positive result with the same culture, the infection status of the HeLa cells can be assured.

### Example 8

### Testing of Additional Primers with Mismatch Relative to E. coli 16S rRNA Gene Sequence

The effect of mismatches with E. coli 16S rRNA situated within the 3′-terminal nucleotides of a primer sequence was further investigated using the primer mtri1-2 shown below. This primer, shown below aligned with the mtri1A primer and the corresponding Mycoplasma and E. coli 16S rRNA sequences, has a two base mismatch at positions-3 and -4 from the 3′ end relative to the corresponding E. coli 16S rRNA sequence (the primer defines a sequence shifted two nucleotides 3′ of the sequence defined by the mtri1A primer). PCR analysis using this primer in analyses with samples from the species of Mycoplasma used above demonstrated that it detects Mycoplasma with the same species specificity as the assays using the mtri1A primer. Further, the mtri1-2 primer failed to direct the amplification of a band from E. coli nucleic acid.

Alignment:

| | |
|---|---|
| mtri1A | agaaagcgatggctaactatg |
| mtri1-2 | ...................tg |
| Msa | ....................... |
| Mar | ....................... |
| Mor | ....................... |
| Mho | ....................... |
| Ala (mtri1B) | .at....ccc............. |
| Mfe | n......a.c............. |
| Mpi | .at...ta.c.a..........t |
| Mhy | .........c...a......... |
| Eco | .ag....acc.........cc.. |

All patents, patent applications, and published references cited herein are hereby incorporated by reference in their entirety. While this invention has been particularly shown and described with references to preferred embodiments thereof, it will be understood by those skilled in the art that various changes in form and details may be made therein without departing from the scope of the invention encompassed by the appended claims.

### REFERENCES

Barile, M. F. (1979). The Mycoplasmas. M. F. Barile and S. Razin. New York, Academic Press: 425-474.
Corless, C. E., M. Guiver, R. Borrow, V. Edwards-Jones, E. B. Kaczmarski, and A. J. Fox. (2000). Contamination and Sensitivity Issues with Real-Time Universal 16S rRNA PCR. J of Clin Microb 38(5): 1747-1752.
DelGuidice, R. A. and H. E. Hopps (1978). Mycoplasma infection of cell cultures. G. J. McGarrity, D. G. Murphy and W. W. Nichols. New York, Plenum Press: 57-69.
DuBois, D. B., C. R. WalkerPeach, M. M. Winkler, and B. L. Pasloske. (1999) Standards for PCR Assays in PCR Applications, Academic Press.
Kong, F., G. James, et al. (2001). Species-specific PCR for identification of common contaminant mollicutes in cell culture. Appl Environ Microbiol 67(7): 3195-200.
McGarrity, G. J. and Kotani (1985). The Mycoplasmas. M. F. Barile and S. Razin. New York, Academic Press: 353-390.
McKenzie, Doug and Robert Buchner (1992). Detection of Mycoplasma Infection in Tissue Culture Cell Lines by Polymerase Chain Reaction. Stratagene Internal Publication.
McKenzie, Doug and Robert Buchner (1996). PCR-based Mycoplasma Detection Kit. Stratagene Internal Publication.
Neimark, H. C. and C. S. Lange (1990). Pulse-field electrophoresis indicates full-length Mycoplasma chromosomes range widely in size. Nucleic Acids Res 18(18): 5443-5448.
Tang, J., M. Hu, et al. (1999). “Primer mixture enhances PCR detection of Mycoplasma/Acholeplasma contaminants in cell cultures.” In Vitro Cell Dev Biol Anim 35(1): 1-3.

Tables

TABLE 1

Nucleotide sequences for amplification primers,
IAC primers, and IAC

| | | |
|---|---|---|
| sequence | Length | Sequence |
| name | (nt) | (5′→3′) |

| | | | |
|---|---|---|---|
| mtri1A | 21 | AGAAAGCGATGGCTAACTATG | (SEQ ID NO: 1) |
| mtri1B | 21 | AATAAGCCCCGGCTAACTATG | (SEQ ID NO: 3) |
| mtrilD | 28 | CCATTTGAATAAGTAACGACTAACTATG | (SEQ ID NO: 4) |
| mtri2short | 16 | GCGTGGACTACCAGGG | (SEQ ID NO: 2) |
| nic1A | 41 | AGAAAGCGATGGCTAACTATGAAGCCTGATCATTT | (SEQ ID NO: 6) |
| | | CCATGT | |
| nic1B | 37 | GCGTGGACTACCAGGGAGGCTCCAGCATCTGTACT | (SEQ ID NO: 7) |
| | | CT | |
| IAC | 495 | AGAAAGCGATGGCTAACTATGAAGCCTGATCATTT | (SEQ ID NO: 5) |
| | | CCATGTCATACTGAGAAAGTCCCCACCCTTCTCTG | |
| | | AGCCTCAGTTTCTCTTTTTATAAGTAGGAGTCTGGA | |
| | | GTAAATGATTTCCAATGGCTCTCATTTCAATACAA | |
| | | AATTTCCGTTTATTAAATGCATGAGCTTCTGTTACT | |
| | | CCAAGACTGAGAAGGAAATTGAACCTGAGACTCA | |
| | | TTGACTGGCAAGATGTCCCCAGAGGCTCTCATTCA | |
| | | GCAATAAAATTCTCACCTTCACCCAGGCCCACTGA | |
| | | GTGTCAGATTTGCATGCACTAGTTCACGTGTGTAA | |
| | | AAAGGAGGATGCTTCTTTCCTTTGTATTCTCACATA | |
| | | CCTTTAGGAAAGAACTTAGCACCCTTCCCACACAG | |
| | | CCATCCCAATAACTCATTTCAGTGACTCAACCCTTG | |
| | | ACTTTATAAAAGTCTTGGGCAGTATAGAGCAGAGA | |
| | | TTAAGAGTACAGATGCTGGATCCGCCCTGGTAGTC | |
| | | CACGC | |

TABLE 2

Multiple sequence alignment of primers mtri1A
and mtri2short with target

| | |
|---|---|
| species | sequence |

| | | |
|---|---|---|
| mtri1A | 5′AGAAAGCGATGGCTAACTATG | (SEQ ID NO: 1) |
| Msa | ..................... | |
| Mar | ..................... | |
| Mor | ..................... | |
| Mho | ..................... | |
| Ala | .AT....CCC........... | |
| Mfe | N......A.C........... | |
| Mpi | .AT...TA.C.A......... | |
| Mhy | .........C...A....... | |
| Eco | .AG....ACC.........CC | |
| mtri2short | 5′GCGTGGACTACCAGGG | (SEQ ID NO: 2) |
| Msa | ................ | |
| Mar | ................ | |
| Mor | ................ | |
| Mho | ................ | |
| Ala | ................ | |
| Mfe | ................ | |
| Mpi | .T.........T.... | |
| Mhy | ................ | |
| Eco | ................ | |
| 3′ Matched | GGCTAACTATGTGCCAGCAGC | |
| Primer | | |
| (forward) | | |
| Eco | C........CC.......... | |

Shown are multiple sequence alignments of primers mtri1A and mtri2short with their published homologous sequences (GenBank). Dots represent reported nucleotides that are identical to the primer. Mismatches are shown as letters. Species are denoted as Ala, Acholeplasma laidlawii, Eco, Esherichia coli K12, Mar, Mycoplasma arginini, Mfe, M. fermentans, Mho, M. hominis, Mhy, M. hyorhinis, Mor, M. orale, Mpi, M. pirum, Msa, M. salivarium. The 3′ Matched forward primer used in FIG. 3 is shown aligned with the E. coli 16S region for comparison.

TABLE 3
Comparison of the sensitivity of the 16S Primer Set with the Mycoplasma
PIus ™ primer set (Stratagene).
New Primer Set (mtrilA/mtrilB/mtrilD/mtrl2short) 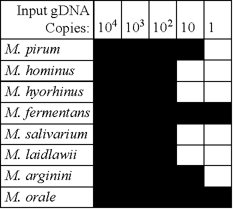
Mycoplasma Plus ™ Primer Set

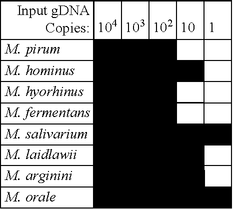

1. A method of increasing the specificity of a PCR-based bacterial assay, the method comprising
aligning a chosen non-E. coli bacterial target nucleic acid sequence with a homologous E. coli nucleic acid sequence;
selecting a PCR primer sequence such that it comprises a sequence perfectly complementary in its three 3′-terminal nucleotides to the chosen non-E. coli bacterial target nucleic acid sequence, and one or more mismatches, in its three 3′-terminal nucleotides, to said homologous E. coli nucleic acid sequence; and
performing PCR using said PCR primer sequence in a PCR-based bacterial assay.

2. The method of claim 1, wherein said primer sequence comprises two or more mismatches in its three 3′-terminal nucleotides, to said corresponding E. coli nucleic acid sequence.

3. The method of claim 1, wherein said primer sequence comprises three mismatches in its three 3′-terminal nucleotides, to said corresponding E. coli nucleic acid sequence.

4. A composition comprising an oligonucleotide primer that hybridizes under standard conditions to a nucleic acid sequence comprised by a Mycoplasma 16S rRNA gene, wherein the 3′-terminal two nucleotides of the primer are selected so as not to base pair with template if the oligonucleotide primer cross-hybridizes with an E. coli 16S rRNA gene template.

5. The composition of claim 4 wherein said primer hybridizes to a 16S rRNA gene sequence from at least two species of Mycoplasma.

6. The composition of claim 4 wherein said primer hybridizes to a 16S rRNA gene sequence from at least three species of Mycoplasma.

7. The composition of claim 4 wherein said primer hybridizes to a 16S rRNA gene sequence from at least four species of Mycoplasma.

8. The composition of claim 4 wherein said primer hybridizes to a 16S rRNA gene sequence from at least five species of Mycoplasma.

9. The composition of claim 4 wherein said primer hybridizes to a 16S rRNA gene sequence from at least six species of Mycoplasma.

10. The composition of claim 4 wherein said primer hybridizes to a 16S rRNA gene sequence from at least seven species of Mycoplasma.

11. The composition of claim 4 wherein said primer hybridizes to a 16S rRNA gene sequence from at least eight species of Mycoplasma.

12. The composition of claim 4 that comprises at least two oligonucleotide primers that hybridize to opposite strands of a nucleic acid sequence comprised by a Mycoplasma 16S rRNA gene, wherein the 3′-terminal two nucleotides of at least one of the primers are selected so as not to base pair with template if the at least one oligonucleotide primer cross-hybridizes with an E. coli 16S rRNA gene template.

13. The composition of claim 4, further comprising an internal amplification control template.

14. The composition of claim 13 wherein said internal amplification control template has the sequence of SEQ ID NO: 5 or the complement thereof.

15. The composition of claim 4 wherein said oligonucleotide primer has a sequence selected from the group consisting of (SEQ ID Nos 1-4).

16. A kit comprising a composition of claim 4.

17. The kit of claim 16, further comprising an internal amplification control template nucleic acid.

18. The kit of claim 17 wherein said internal amplification control template has the sequence of SEQ ID NO 5 or the complement thereof.

19. The kit of claim 16 further comprising a template-dependent nucleic acid extending enzyme.

20. The kit of claim 16 further comprising a uracil DNA glycosylase enzyme.

21. An isolated nucleic acid consisting of the sequence of any one of SEQ ID NOs 1-4 or the respective complement thereof.

22. An isolated nucleic acid comprising the sequence of SEQ ID NO: (5) or the complement thereof.

23. A kit comprising an isolated nucleic acid of claim 21.

24. The kit of claim 23 further comprising an internal amplification control nucleic acid template.

25. The kit of claim 24 wherein said internal amplification control nucleic acid template comprises the sequence of SEQ ID NO (5) or the complement thereof.

26. The kit of claim 23 further comprising a template-dependent nucleic acid extending enzyme.

27. The kit of claim 23 further comprising a uracil DNA glycosylase enzyme.

28. A method of detecting the presence of a Mycoplasma species in a sample, the method comprising:
forming a reaction mixture comprising the sample and a set of oligonucleotide primers that hybridize under standard conditions to a nucleic acid sequence comprised by a Mycoplasma 16S rRNA gene, wherein the 3′-terminal nucleotide of one or more of said set of primers is selected so as not to base pair with an E. coli 16S rRNA gene template if the oligonucleotide primer cross-hybridizes with said E. coli 16S rRNA gene template;
extending said primers; and
detecting extension products of said primers, wherein the presence of an extension product is indicative of the presence of a Mycoplasma species in said sample.

29. The method of claim 28 wherein the 3′-terminal two nucleotides of one or more of said set of primers are selected so as not to base pair with an E. coli 16S rRNA gene template if said one or more of said set of oligonucleotide primers cross-hybridizes with said E. coli 16S rRNA gene template.

30. The method of claim 28 wherein the step of extending said primers comprises polymerase chain reaction (PCR) amplification.

31. The method of claim 30 wherein the reaction mixture further comprises an internal amplification control template, wherein the product of amplification of the internal amplification control template is detectably different in size or sequence than the product of amplification of Mycoplasma 16S rRNA species amplified in said sample.

32. The method of claim 31 wherein said internal amplification control comprises a nucleic acid template comprising 5′ and 3′ regions that hybridize with corresponding regions of a 16S rRNA gene sequence from one or more Mycoplasma species under standard conditions, flanking a central region of non-16S rRNA gene sequence, wherein said 5′ and 3′ regions hybridize to oligonucleotide primers in said primer set.

33. The method of claim 31 wherein said internal amplification control template comprises the sequence of SEQ ID NO. 5.

34. The method of claim 28 further comprising the step, before the step of extending said primers, of contacting said reaction mixture with a uracil DNA glycosylase enzyme.

35. The method of claim 28 wherein said reaction mixture comprises dUTP.

36. The method of claim 28 wherein the step of extending said primers is performed in the presence of dUTP, and wherein said extending results in the incorporation of dUTP into an extension product.

37. The method of claim 28 wherein the step of detecting extension products comprises gel electrophoresis.

38. The method of claim 28 wherein the set of oligonucleotide primers comprises a primer selected from the group consisting of: (SEQ ID Nos: 1-4).

39. The method of claim 28 wherein the set of oligonucleotide primers comprises SEQ ID Nos: 1 & 2.

40. The method of claim 28 wherein the set of oligonucleotide primers consists of SEQ ID Nos: 1-4.

41. The method of claim 28 wherein the set of oligonucleotide primers detects the presence of Mycoplasma species including Acholeplasma laidlawii, Mycoplasma arginini, M. fermentans, M. hominis, M. hyorhinis, M. orale, M. salivarium, and M. pirum.

42. A method for detecting the presence of a Mycoplasma species in a sample, the method comprising:
contacting the sample with a set of oligonucleotide primers that hybridize to a 16S rRNA gene sequence in one or more Mycoplasma species, the set of oligonucleotide primers comprising SEQ ID NO: 2;
extending at least one of said primers; and
detecting an extension product, wherein the presence of an extension product indicates the presence of a Mycoplasma species in said sample.

43. The method of claim 42, wherein said set of oligonucleotide primers further comprises a primer selected from the group consisting of SEQ ID NOs 1, 3 and 4.

44. The method of claim 42, wherein said set of oligonucleotide primers further comprises oligonucleotide primers of SEQ ID NOs 1 & 3.

45. The method of claim 42, wherein said set of oligonucleotide primers further comprises oligonucleotide primers of SEQ ID NOs 1 & 4.

46. The method of claim 42, wherein said set of oligonucleotide primers further comprises oligonucleotide primers of SEQ ID NOs 1, 3 & 4.

47. The method of claim 42 wherein for at least one member of the set of oligonucleotide primers, the 3′-terminal nucleotide is selected such that it does not base pair with an E. coli 16S rRNA gene template if said member cross-hybridizes with an E. coli 16S rRNA gene template.

48. The method of claim 47 wherein for at least one member of the set of oligonucleotide primers, the 3′-terminal two nucleotides are selected such that they do not base pair with an E. coli 16S rRNA gene template if said member cross-hybridizes with an E. coli 16S rRNA gene template.

49. The method of claim 42 wherein the step of extending said primers comprises polymerase chain reaction (PCR) amplification.

50. The method of claim 42 further comprising the step, either before, or concurrent with the step of contacting the sample with a set of oligonucleotides, of adding an internal control template to said sample.

51. The method of claim 50 wherein the extension product of said internal control template is detectably different in size or sequence from any extension product resulting from extension of said set of primers on a Mycoplasma 16S rRNA gene nucleic acid template.

52. The method of claim 42 wherein said internal amplification control template comprises the sequence of SEQ ID NO. 5.

53. The method of claim 42 further comprising the step, before the step of extending said primers, of contacting said reaction mixture with a uracil DNA glycosylase enzyme.

54. The method of claim 42 wherein said reaction mixture comprises dUTP.

55. The method of claim 42 wherein the step of extending said primers is performed in the presence of dUTP, and wherein said extending results in the incorporation of dUTP into an extension product.

56. The method of claim 42 wherein the step of detecting extension products comprises gel electrophoresis.

57. The method of claim 42 wherein the set of oligonucleotide primers detects the presence of Mycoplasma species including Acholeplasma laidlawii, Mycoplasma arginini, M. fermentans, M. hominis, M. hyorhinis, M. orale, M. salivarium, and M. pirum.

58. A method for detecting the presence of a Mycoplasma species in a sample, the method comprising:
contacting the sample with a set of oligonucleotide primers that hybridize to a 16S rRNA gene sequence in one or more Mycoplasma species, the set of oligonucleotide primers comprising SEQ ID NO. 1.
extending at least one of said primers;
detecting an extension product, wherein the presence of an extension product indicates the presence of a Mycoplasma species in said sample.

59. The method of claim 58, wherein said set of oligonucleotide primers further comprises a primer selected from the group consisting of SEQ ID NOs 2, 3, & 4.

60. The method of claim 58, wherein said set of oligonucleotide primers further comprises oligonucleotide primers of SEQ ID NOs 3 & 2.

61. The method of claim 58, wherein said set of oligonucleotide primers further comprises oligonucleotide primers of SEQ ID NOs 4 & 2.

62. The method of claim 58, wherein said set of oligonucleotide primers further comprises oligonucleotide primers of SEQ ID Nos 2, 3 & 4.

63. The method of claim 58 wherein for at least one member of the set of oligonucleotide primers, the 3′-terminal nucleotide is selected such that it does not base pair with an E. coli 16S rRNA gene template if said member cross-hybridizes with an E. coli 16S rRNA gene template.

64. The method of claim 63 wherein for at least one member of the set of oligonucleotide primers, the 3′-terminal two nucleotides are selected such that they do not base pair with an E. coli 16S rRNA gene template if said member cross-hybridizes with an E. coli 16S rRNA gene template.

65. The method of claim 58 wherein the step of extending said primers comprises polymerase chain reaction (PCR) amplification.

66. The method of claim 58 further comprising the step, either before, or concurrent with the step of contacting the sample with a set of oligonucleotides, of adding an internal control template to said sample.

67. The method of claim 66 wherein the extension product of said internal control template is detectably different in size or sequence from any extension product resulting from extension of said set of primers on a Mycoplasma 16S rRNA gene nucleic acid template.

68. The method of claim 58 wherein said internal amplification control template comprises the sequence of SEQ ID NO. 5.

69. The method of claim 58 further comprising the step, before the step of extending said primers, of contacting said reaction mixture with a uracil DNA glycosylase enzyme.

70. The method of claim 58 wherein said reaction mixture comprises dUTP.

71. The method of claim 58 wherein the step of extending said primers is performed in the presence of dUTP, and wherein said extending results in the incorporation of dUTP into an extension product.

72. The method of claim 58 wherein the step of detecting extension products comprises gel electrophoresis.

73. The method of claim 58 wherein the set of oligonucleotide primers detects the presence of Mycoplasma species including Acholeplasma laidlawii, Mycoplasma arginini, M. fermentans, M. hominis, M. hyorhinis, M. orale, M. salivarium, and M. pirum.

74. A method for detecting the presence of a Mycoplasma species in a sample, the method comprising:
contacting the sample with a set of oligonucleotide primers that hybridize to a 16S rRNA gene sequence in one or more Mycoplasma species, the set of oligonucleotide primers comprising SEQ ID NO. 3;
extending at least one of said primers; and
detecting an extension product, wherein the presence of an extension product indicates the presence of a Mycoplasma species in said sample.

75. The method of claim 74, wherein said set of oligonucleotide primers further comprises a primer selected from the group consisting of SEQ ID NOs 1, 2 & 4.

76. The method of claim 74, wherein said set of oligonucleotide primers further comprises oligonucleotide primers of SEQ ID NOs 1 & 2.

77. The method of claim 74, wherein said set of oligonucleotide primers further comprises oligonucleotide primers of SEQ ID NOs 4 & 2.

78. The method of claim 74, wherein said set of oligonucleotide primers further comprises oligonucleotide primers of SEQ ID NOs 1, 2 & 4.

79. The method of claim 74 wherein for at least one member of the set of oligonucleotide primers, the 3′-terminal nucleotide is selected such that it does not base pair with an E. coli 16S rRNA gene template if said member cross-hybridizes with an E. coli 16S rRNA gene template.

80. The method of claim 79 wherein for at least one member of the set of oligonucleotide primers, the 3′-terminal two nucleotides are selected such that they do not base pair with an E. coli 16S rRNA gene template if said member cross-hybridizes with an E. coli 16S rRNA gene template.

81. The method of claim 74 wherein the step of extending said primers comprises polymerase chain reaction (PCR) amplification.

82. The method of claim 74 further comprising the step, either before, or concurrent with the step of contacting the sample with a set of oligonucleotides, of adding an internal control template to said sample.

83. The method of claim 82 wherein the extension product of said internal control template is detectably different in size or sequence from any extension product resulting from extension of said set of primers on a Mycoplasma 16S rRNA gene nucleic acid template.

84. The method of claim 74 wherein said internal amplification control template comprises the sequence of SEQ ID NO. 5.

85. The method of claim 74 further comprising the step, before the step of extending said primers, of contacting said reaction mixture with a uracil DNA glycosylase enzyme.

86. The method of claim 74 wherein said reaction mixture comprises dUTP.

87. The method of claim 74 wherein the step of extending said primers is performed in the presence of dUTP, and wherein said extending results in the incorporation of dUTP into an extension product.

88. The method of claim 74 wherein the step of detecting extension products comprises gel electrophoresis.

89. The method of claim 74 wherein the set of oligonucleotide primers detects the presence of Mycoplasma species including Acholeplasma laidlawii, Mycoplasma arginini, M. fermentans, M. hominis, M. hyorhinis, M. orale, M. salivarium, and M. pirum.

90. A method for detecting the presence of a Mycoplasma species in a sample, the method comprising:
contacting the sample with a set of oligonucleotide primers that hybridize to a 16S rRNA gene sequence in one or more Mycoplasma species, the set of oligonucleotide primers comprising SEQ ID NO. 4;
extending at least one of said primers; and
detecting an extension product, wherein the presence of an extension product indicates the presence of a Mycoplasma species in said sample.

91. The method of claim 90 further comprising the step, either before, or concurrent with the step of contacting the sample with a set of oligonucleotides, of adding an internal control template to said sample.

92. The method of claim 91 wherein the extension product of said internal control template is detectably different in size or sequence from any extension product resulting from extension of said set of primers on a Mycoplasma 16S rRNA gene nucleic acid template.

93. The method of claim 90, wherein said set of oligonucleotide primers further comprises a primer selected from the group consisting of SEQ ID NOs 1, 2 & 3.

94. The method of claim 90, wherein said set of oligonucleotide primers further comprises oligonucleotide primers of SEQ ID NOs 1 & 2.

95. The method of claim 90, wherein said set of oligonucleotide primers further comprises oligonucleotide primers of SEQ ID NOs 3 & 2.

96. The method of claim 90, wherein said set of oligonucleotide primers further comprises oligonucleotide primers of SEQ ID Nos 1, 2 & 3.

97. The method of claim 90 wherein for at least one member of the set of oligonucleotide primers, the 3′-terminal nucleotide is selected such that it does not base pair with an E. coli 16S rRNA gene template if said member cross-hybridizes with an E. coli 16S rRNA gene template.

98. The method of claim 97 wherein for at least one member of the set of oligonucleotide primers, the 3′-terminal two nucleotides are selected such that they do not base pair with an E. coli 16S rRNA gene template if said member cross-hybridizes with an E. coli 16S rRNA gene template.

99. The method of claim 90 wherein the step of extending said primers comprises polymerase chain reaction (PCR) amplification.

100. The method of claim 90 wherein said internal amplification control template comprises the sequence of SEQ ID NO. 5.

101. The method of claim 90 further comprising the step, before the step of extending said primers, of contacting said reaction mixture with a uracil DNA glycosylase enzyme.

102. The method of claim 90 wherein said reaction mixture comprises dUTP.

103. The method of claim 90 wherein the step of extending said primers is performed in the presence of dUTP, and wherein said extending results in the incorporation of dUTP into an extension product.

104. The method of claim 90 wherein the step of detecting extension products comprises gel electrophoresis.

105. The method of claim 90 wherein the set of oligonucleotide primers detects the presence of Mycoplasma species including Acholeplasma laidlawii, Mycoplasma arginini, M. fermentans, M. hominis, M. hyorhinis, M. orale, M. pirum, and M. salivarium.
